(19) United States
(12) Patent Application Publication
(10) Pub. No.: US 2021/0183505 A1
(43) Pub. Date: Jun. 17, 2021

(54) SYSTEM AND METHOD FOR CONNECTING PATIENTS, MEDICAL SERVICE PROVIDERS, AND MEDICAL INSURANCE PROVIDERS

(71) Applicant: ORBIT HEALTHCARE, INC., East Brunswick, NJ (US)

(72) Inventor: Krishna VELAGA, East Brunswick, NJ (US)

(73) Assignee: ORBIT HEALTHCARE, INC., East Brunswick, NJ (US)

(21) Appl. No.: 17/166,356

(22) Filed: Feb. 3, 2021

Related U.S. Application Data
(63) Continuation-in-part of application No. 15/462,132, filed on Mar. 17, 2017.

(51) Int. Cl. G16H 40/20; G06Q 30/00; G16H 10/60; G06Q 40/08; G06Q 10/10; G06Q 20/14; G16H 80/00; G06F 16/9535

(52) U.S. Cl. CPC G16H 40/20; G06Q 30/0185; G16H 10/60; G06F 16/9535; G06Q 10/1057; G06Q 20/14; G16H 80/00; G06Q 40/08

(57) ABSTRACT

Methods and systems are provided for managing healthcare information. A data retrieval application includes a fraud detection engine that monitors medical claims of a user submitted to a third party insurer, a database of a plurality of healthcare providers, and a database of one or more users. The data retrieval application allows remote access to a centralized information store for the user, a primary healthcare provider, and the third party insurer. The centralized information store houses the pre-visit identifying documentation for the user and allows for reusability of the information.

### CROSS-REFERENCE TO RELATED APPLICATIONS SECTION

This application is a United States Continuation-in-Part (CIP) and Non-Provisional Patent Application that claims priority to U.S. Non-Provisional patent application Ser. No. 15/462,132 filed on Mar. 17, 2017 and published as U.S. Published Patent Application No. 2018/0268106 A1 on Sep. 20, 2018, the entire contents of which are hereby incorporated by reference in their entirety.

### FIELD OF THE EMBODIMENTS

This invention relates to consumer data management and, in particular, to facilitating communication between patients, healthcare providers, and relevant third parties.

### BACKGROUND OF THE EMBODIMENTS

Receiving medical treatment is not a simple task. Both before and after a medical procedure is performed, there is information that must be communicated between the patient, the healthcare provider(s), and any relevant third parties, such as insurance companies. Each of these parties requires information from the other two to ensure that a correct procedure is performed, and that the procedure can be properly paid for.

From the standpoint of patients, there exists various websites that enable users to search for healthcare providers, but these websites offer a way for the patients to communicate with the providers. There are multiple portals, websites, etc. that patients have to go through in order to both discuss issues with their healthcare providers and to determine if their insurance covers any procedures that are to be performed. This lack of easily accessible communicative means thus makes it difficult to exchange any pre-visit documentation without having to carry every required document and complete waiting room forms.

From the standpoint of healthcare providers, the lack of pre-visit communication with the patients decreases efficiency at doctors' offices, causes delays in appointments, and thus increase their cost of operating their practices.

From the standpoint of third party players, such as insurance companies, there is a lack of visibility into provider services from the patient's point of view until a claim adjustment is complete. This can lead to fraudulent claims.

For at least these reasons, there is a need for a system and method for managing healthcare information that enables all of these parties to adequately interact with one another.

Examples of related art are described below:

U.S. Pat. No. 7,188,151 pertains to a system and method for network-based monitoring of physiological data. At least one patient-side device collects physiological data from a patient. A provider-side device receives the data from at least one patient-side device via a wide area network. An engine communicates with at least one patient-side device and the provider-side device. The engine manages transmission of the data from the patient-side device to the provider-side device.

U.S. Pat. No. 7,752,060 pertains to an Internet-based system that involves a database and search capabilities for connecting patients with healthcare providers, e.g., physicians, hospitals, nursing homes, treatment facilities, etc., and further enables such providers to reach patients with whom they may not otherwise come into contact. A patient may access the healthcare provider information through a search conducted using a search engine, such as Google, Yahoo, etc. Alternatively, a patient may access the company Web site's predetermined Web page that provides search capabilities on its database. A patient may research a healthcare provider based on criteria specified by the patient. Information provided to the patient may be in the form of a report, profile, ratings, etc., including patient-provided information, physician-verified information, and information verified by an independent third party. The verified information and ratings provided by the Web site enable patients to differentiate among healthcare providers and thereby select the provider that best meets their individual needs.

U.S. Pat. No. 8,719,052 pertains to an Internet-based system that involves a database and search capabilities for connecting patients with healthcare providers, e.g., physicians, hospitals, nursing homes, treatment facilities, etc., and further enables such providers to reach patients with whom they may not otherwise come into contact. A patient may access the healthcare provider information through a search conducted using a search engine, such as Google, Yahoo, etc. Alternatively, a patient may access the company Web site's predetermined Web page that provides search capabilities on its database. A patient may research a healthcare provider based on criteria specified by the patient. Information provided to the patient may be in the form of a report, profile, ratings, etc., including patient-provided information, physician-verified information, and information verified by an independent third party. The verified information and ratings provided by the Web site enable patients to differentiate among healthcare providers and thereby select the provider that best meets their individual needs.

U.S. Patent Application No. 2002/0123909 pertains to methods to allow for patient/consumers to collect, store, organize, password protect, and share personal health, medical, dental, and pharmacy data without regard to healthcare provider systems are provided. Application software necessary for complete functionality is contained on a portable, pocket sized, re-write enabled compact disk. Application software necessary for providers (doctors, dentists, hospitals, pharmacies) to view, print, and modify data points is contained on the compact disk. Communication software for the automatic updating of patient information by provider billing/management systems is contained on the compact disk. All software functions necessary for the user/patient and providers to collect, store, update, organize, and share patient information is contained within the application software imbedded on the compact disk. The compact disk therefore is the medium for consumers to collect, organize, store, and share their patient information with providers authorized by the consumer thus optimizing the health history information available to providers. Providing this information to care givers at the time of service serves to improve care and protect the consumer from endangerment due to lack of health history information, care conflicts, and/or drug-drug interactions. Consumer/patient control of access to the application further serves to protect against breech of privacy and confidentiality.

U.S. Patent Application No. 2002/0128883 pertains to a system and method for on-line collaboration and advanced management of insurance claims with the direct sharing of claim data and information in real time. Information and data is shared between insurers and service providers and any other parties deemed necessary for enhanced resolution of the claim.

U.S. Patent Application No. 2002/0169638 pertains to an integrated, electronic patient record system and method for providing real time point of care evaluation, management, testing, data entry, billing, and treatment via wireless, paperless medical care processing. The system includes automatic data entry from clinical equipment, structured and non-structured examination protocol templates, synoptic patient chart view, cross-linked billing and diagnosis, digital generation of medical claims, point of care data entry and laboratory results. The present invention facilitates the generation of computerized prescriptions and transfer of same and/or electronic medical records to the patient or other medical entity for which the patient has provided consent where required.

U.S. Patent Application No. 2003/0028402 pertains to a method and system for electronically maintaining medical records and to facilitate availability and use of information relating to patients, as well as other information used in the operation of a medical facility. The system stores information such as patient charts, medical histories of patients, insurance information, including documentation requirements of insurance companies, timelines for calendaring events, and dictionaries of commonly or repetitively used information. The system permits inputting of information relating to a patient encounter or appointment with a patient and inputting information on business related contacts, including information on patients, pharmacies, physicians and insurance companies. The system further facilitates tracking of patients and their medical status, prescription writing, printing of reports, and preparation of insurance forms, including electronic claim forms.

U.S. Patent Application No. 2004/0006496 pertains to a system and method for monitoring and managing, in real-time, interactions between a healthcare provider, a patient and an insurer is disclosed. The system and method comprises of a virtual or electronic waiting room. Management and monitoring of patient interactions and transactions is achieved by storing attributes associated with a patient and linking these attributes to a virtual representation of the patient, which may be accessed and manipulated via the electronic or virtual waiting room.

U.S. Patent Application No. 2004/0210458 pertains to methods and platforms for enhancing collaboration and communication between a patient and his healthcare team. A personal health record is created for a patient and maintained by a service provider. The health record is updated with self-monitored or remote device readings. These readings are sent, in a secure format that insures patient privacy, to the service provider and inserted into a health record via a computer connected to the Internet or via a telephone line without the use of a computer, i.e., by directly connecting an intermediate device to a phone outlet. Other health and wellness data may be written to the health record via a computer or via conventional means. Personal health records are created with minimal effort from healthcare professionals and patients. Third-party companies in the healthcare industry, such as insurance companies and pharmaceutical companies, can sponsor programs encouraging the creation of personal health records and, in turn, receive depersonalized summary information from the service provider to study healthcare trends. By enhancing collaboration between patients and their healthcare teams, patients are more likely to improve their health conditions, particularly chronic conditions, and reduce healthcare costs.

U.S. Patent Application No. 2004/0220829 pertains to an online consultation platform that provides an interactive patient interview, produces a succinct message to the provider, and a prompt response to the patient's query; online prescribing and refills and transmission of the prescription; streamlined messaging between patient and provider employs via specialized message types; practice and workflow management for the provider that includes specialized message types, customizable routing, and role-based permissions; customizable practice web sites for registered providers, wherein patients can visit to access online services; broadcast of patient education materials customized and automatically distributed to targeted patient groups; and integrated charging and collections, determination of eligibility for coverage, and reimbursement.

U.S. Patent Application No. 2006/0277075 pertains to a Physician To Patient (P2P) network system, a private & secure infrastructure for independently practicing physicians and patients for real-time electronic communication & transfer of patient health information is disclosed by the invention. The invention also discloses an efficient and natural method for creation of Electronic Medical Records by physicians in their own handwriting. The P2P network system utilizes a plurality of devices and components defined by the invention, and custom programming to integrate all equipment, devices and components of the network system. The invention also discloses a highly targeted method of advertising, the One2One Advertising, for healthcare product manufacturers to reach physicians and patients. A number of healthcare related business processes, currently executed manually are performed automatically by P2P network system software. The invention will improve the quality of services to patients, and reduce the overhead cost of the medical offices, and the healthcare industry.

U.S. Patent Application No. 2007/0299776 pertains to the use of a real time transmitted encrypted identification system to verify patient ID, location, time and medical service provider identification helps to prevent fraudulent insurance claims. The system may use a networked laptop computer having identity capturing systems such as a bar code reader, magnetic card reader, smartcard reader, RF transponder, fingerprint capture, signature capture, photo image capture or facial recognition software for the patient, the medical service provider, and may have a GPS system, or other location system, to ensure that the ID capture occurs at the authorized location. Insurance companies may provide their authorized medical providers with the networked laptop, or other ID capture device, and control the verification system within the single company, or may be customers of a verification center that handles all or some of the insurance payors in an area.

U.S. Patent Application No. 2012/0130742 pertains to a method and system to enable an internet-based, advanced, electronic communication service between an established patient, already in a previously established doctor-patient relationship, and his/her doctor(s) to facilitate participation in an electronic consultation via an electronic communication connection. The method and system includes initiating connection with a central computer by the patient- and doctor-users; collecting and recording user entered information; creating a unique identifier for each user; and registering the user; collecting and recording medical record information for the patient-user from the doctor-user; creating an electronic relationship between the doctor-user and patient-user; validating the relationship; if said validating step is true, then: opening an electronic connection between the doctor-user, patient-user and computer; transferring question data from the patient-user to the doctor-user through the computer and transferring answer data from the doctor-user back to the patient-user through the computer; and storing the data on the computer.

U.S. Patent Application No. 2012/0232929 pertains to a method, an apparatus, and a computer program product for accessing electronic medical records are provided in which a portable computing device uniquely associated with a user authenticates an identification of the user and automatically retrieves information corresponding to the user from electronic healthcare records systems using the identification. The retrieved information may be combined with other information and electronically delivered to a healthcare provider.

U.S. Patent Application No. 2014/0136233 pertains to a system, method and computer program for generating a Global Personal Health Record Timeline (GPHRT). A plurality of data items and information points associated with at least one relationship between users associated with a single patient, healthcare provider, a group of healthcare providers. Ordered data items are stored in a personal health record, transmitted and shared via a plurality of storage and transmittal technologies such as, (BLE), barcode scanner, and a plurality of other storage and transmittal technologies that will interface with the Global Personal Health Record Timeline platform. These aforementioned embodiments would further be integrated into an Enterprise Resource Planning Electronic Medical Records Software Environment (ERP/EMRSE) which would interface with a Database Repository with a web-based Global Patient Health Record Timeline interface portal, security interface, Customer Resource Management platform, Practice Management platform, e-commerce interface, “HIPPA” compliant security filter.

International Patent Application No. WO 01/93172 A1 pertains to a method of collecting and distributing medical information at a computer server. The method includes the steps of establishing computer network communications between the computer server and a plurality of medical institutions, establishing communication between the computer server and a medical transaction device, creating a database of medical information on the computer server relating to a plurality of patients with information received from the medical institution via the medical transaction device, and providing access to the database of medical information via the medical transaction device. The step of creating a medical database may further comprise the computer server receiving information from the plurality of medical institutions. The step of creating a medical database may also further comprise the steps of formulation a query for medical information, transmitting the query for medical information from the computer server to at least one medical institution, and receiving medical information at the computer server from the queried medical institution.

It is noted that none of the art described above addresses all of the issues that the present invention does.

### SUMMARY OF THE EMBODIMENTS

According to an embodiment of the present invention, a method is provided for managing healthcare information. The method includes the steps of: storing, in a memory, a database of a plurality of healthcare providers and a database of one or more users; enabling a user, using a graphical user interface, to search the database of the plurality of healthcare providers; receiving, from the user, using the graphical user interface, one or more search parameters; returning, to the user, a list of all healthcare providers that satisfy the search parameters; selecting, using the graphical user interface, a healthcare provider, from the list of healthcare providers that satisfy the search parameters, as a primary healthcare provider; storing, in the memory, a patient care history of the user; enabling remote access to the patient care history by the user, the primary healthcare provider, and a third party player using a data retrieval computer application; and enabling communication between the user, the primary healthcare provider, and the third party player using the data retrieval computer application.

According to another embodiment of the present invention, a system is provided for managing healthcare information. The system includes a memory configured to store a data retrieval computer application, a database of a plurality of healthcare providers, a database of one or more users, and a patient care history for each user. The system further includes a graphical user interface. The system additionally includes a processor configured to run the access the data retrieval computer application, the database of a plurality of healthcare providers, and the database of one or more users, wherein when the processor runs the data retrieval computer application, is causes a computing device to: enable a user, using the graphical user interface, to search the database of the plurality of healthcare providers; receive, from the user, using the graphical user interface, one or more search parameters; return, to the user, a list of all healthcare providers that satisfy the search parameters; accept, using the graphical user interface, a healthcare provider, from the list of healthcare providers that satisfy the search parameters, as a primary healthcare provider; enable remote access to the patient care history by the user, the primary healthcare provider, and a third party player using a data retrieval computer application; and enable communication between the user, the primary healthcare provider, and the third party player using the data retrieval computer application.

It is an object of the present invention to provide for the method for managing healthcare information, wherein the search parameters are selected from the group consisting of: medical specialty; name; location; phone number; provider type; languages spoken; and type of insurance accepted.

It is an object of the present invention to provide for the method for managing healthcare information, wherein the database of the plurality of healthcare providers includes identifiable information pertaining to each of the plurality of healthcare providers selected from the group consisting of: medical specialty; name; location; phone number; provider type; languages spoken; and type of insurance accepted.

It is an object of the present invention to provide for the method for managing healthcare information, wherein the database of the one or more users includes identifiable information pertaining to each of the plurality of users selected from the group consisting of: name; address; phone number; insurance type; billing information; employer; languages spoken; and medical history.

It is an object of the present invention to provide for the method for managing healthcare information, wherein the third party player is an insurance provider.

It is an object of the present invention to provide for the method for managing healthcare information, further including coordinating between the primary healthcare provider and the insurance provider, using the data retrieval computer application, to determine an insurance eligibility of the user for one or more medical procedures.

It is an object of the present invention to provide for the method for managing healthcare information, further including enabling remote access to the user's insurance information and employment information to the user, the primary healthcare provider, and the insurance provider to facilitate determination of insurance eligibility.

It is an object of the present invention to provide for the method for managing healthcare information, further including enabling remote access to the user's insurance information and employment information to the user, the primary healthcare provider, and the insurance provider to facilitate payment of one or more medical procedures.

It is an object of the present invention to provide for the method for managing healthcare information, wherein the database of one or more users includes a profile for each user.

It is an object of the present invention to provide for the method for managing healthcare information, wherein the profile for each user includes any medical claims for that particular user.

It is an object of the present invention to provide for the method for managing healthcare information, further including updating the medical claims for a particular user following approval or performance of a medical procedure.

It is an object of the present invention to provide for the method for managing healthcare information, wherein the profile for each user further includes any outstanding payments due to the insurance provider for any medical procedures.

It is an object of the present invention to provide for the method for managing healthcare information, further including automatically paying any outstanding balances using billing information provided by the user.

It is an object of the present invention to provide for the method for managing healthcare information, wherein the enabling communication further includes enabling the user, the primary healthcare provider, and the third party player to communicate using a method selected from the group consisting of: e-mail; text message; video communication; and audio communication.

### BRIEF DESCRIPTION OF THE DRAWINGS

FIG. 1 and FIG. 2 show a flowchart of a method for managing healthcare information, according to an embodiment of the present invention.

FIG. 3 shows a block/flow diagram of a system for managing healthcare information, according to an embodiment of the present invention.

FIG. 4 shows a screenshot of a healthcare provider search engine, according to an embodiment of the present invention.

FIG. 5 shows a screenshot of a healthcare provider search engine, according to an embodiment of the present invention.

FIG. 6 shows a flowchart of a method for scheduling a medical appointment, according to an embodiment of the present invention.

FIG. 7 shows a chart defining various icons used in conjunction with the healthcare provider search engine, according to an embodiment of the present invention.

FIG. 8 shows a flowchart of a method of using a fraud detection bot, according to an embodiment of the present invention.

FIG. 9 shows a block diagram of communication with a centralized information store or vault, according to an embodiment of the present invention.

FIG. 10 shows a schematic diagram of data flow between a patient, a healthcare provider, and a third-party insurer in a system for managing healthcare information, according to an embodiment of the present invention.

FIG. 11 shows a schematic diagram of cloud architecture of a system for managing healthcare information, according to an embodiment of the present invention.

FIG. 12 shows a schematic diagram of architecture/technology stack of a system for managing healthcare information, according to an embodiment of the present invention.

FIG. 13 shows a schematic diagram of patient information and signature capture prior to visiting a healthcare provider, according to an embodiment of the present invention.

FIG. 14 shows a schematic diagram of patient check-in at a kiosk in a waiting room at a healthcare provider, according to an embodiment of the present invention

FIG. 15 shows a schematic diagram of a kiosk implementation in a waiting room at a healthcare provider, according to an embodiment of the present invention

FIG. 16 shows a schematic diagram of kiosk data flow and integration with a centralized information store or vault, according to an embodiment of the present invention.

FIG. 17 depicts a flowchart of a method for reusing information across multiple providers from a kiosk at a healthcare provider, according to an embodiment of the present invention.

FIG. 18 depicts a flowchart of a method for reusing information across multiple providers from an application executed on a mobile device, according to an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

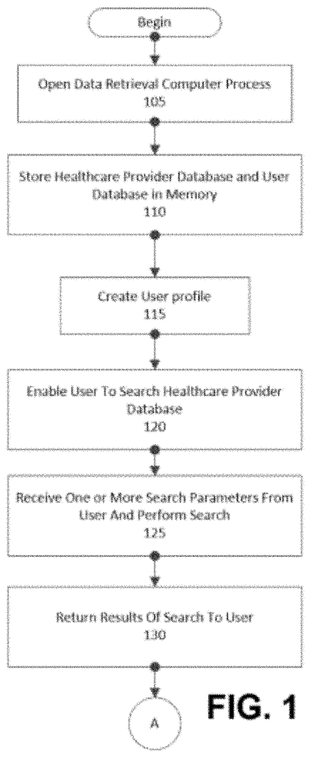

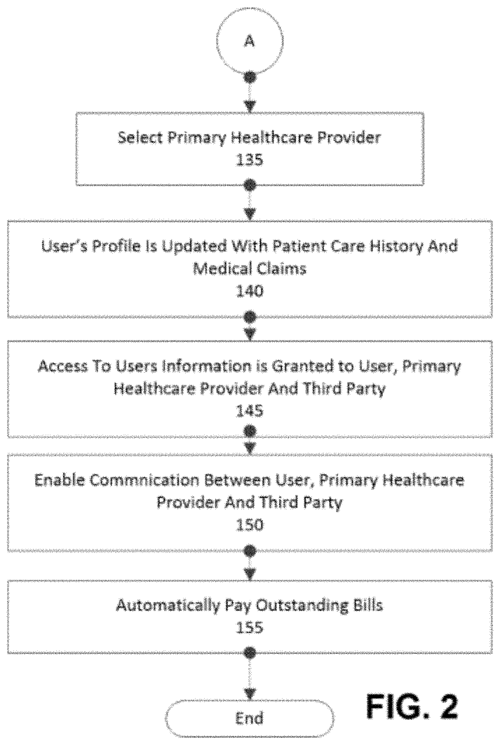

Referring now to FIGS. 1-2, a flowchart of a method for managing healthcare information is illustratively depicted, in accordance with an embodiment of the present invention.

At step 105, a data retrieval computer application is opened. The application may be opened on any suitable computing device such as, but not limited to, a desktop computer, a laptop computer, a tablet computer, a mobile phone, etc. The computer application may cause a computing device to perform any of the following steps.

At step 110, a database of a plurality of healthcare workers and a database of one or more users are stored in a storage medium such as, e.g., a computer memory.

According to an embodiment, the database of the plurality of healthcare providers includes identifiable information pertaining to each of the plurality of healthcare providers. This information may include, for each healthcare provider, the healthcare provider's medical specialty, name, location, phone number, provider type, languages spoken, and/or type of insurance accepted. It is noted that other information related to the healthcare providers may also be stored, while maintaining the spirit of the present invention.

According to an embodiment, the database of the one or more users includes identifiable information pertaining to each of the plurality of users. This information may include, for each user, the user's name, address, phone number, insurance type, billing information, employer, languages spoken, and/or medical history. It is noted that other information related to the plurality of users may also be stored, while maintaining the spirit of the present invention.

At step 115, a user creates a user profile on the application using a graphical user interface. The user's profile includes the user's name, address, phone number, insurance type, billing information, employer, languages spoken, and/or medical history. This information is then stored in the memory in the database of one or more users.

At step 120, a the application enables the user to search through the database of the plurality of healthcare providers using a graphical user interface. According to an embodiment, the user is able to search through the database by narrowing the list of healthcare providers using one or more search parameters. According to an embodiment, the search parameters are predefined. The predefined search parameters may include, e.g., medical specialty, name, location, phone number, provider type, languages spoken, and/or type of insurance accepted. It is noted, however, that other suitable predefined search parameters may also be used, while maintaining the spirit of the present invention.

At step 125, the user inputs one or more the application receives one or more search parameters from the user and performs a search of the database of the plurality of healthcare providers to determine which healthcare providers satisfy the search parameters.

At step 130, all healthcare providers that satisfy the search parameters are returned to the user. According to an embodiment, the user is then able to search through the results to see detailed descriptions of the individual healthcare providers that are stored in the database of the plurality of healthcare providers. If no healthcare providers satisfy the search parameters, the user is notified and informed that a broader search may have to be conducted.

According to an embodiment, the user may designate one or more returned healthcare providers as a “favorite” or other similar designation wherein the user has easy future access to the designated healthcare providers. According to an embodiment, the healthcare providers that the user designates as “favorites” or other similar designation may be viewable by a separate tab and/or search function.

At step 135, the user selects one of the healthcare providers returned in the search as a primary healthcare provider. According to an embodiment, the user may also selected one or more healthcare providers as secondary healthcare providers. According to an embodiment, ad-hoc scheduling is performed in which the user is able to schedule an appointment with one or more healthcare providers directly through the application. According to another embodiment, the user is able to designate a location, date, and/or time at which the user wants to have an appointment. This location, date, and/or time is viewable by one or more healthcare providers, who are able to respond to the user, offering their services for the location, date, and/or time specified. The user may accept the services from any, all, or none of these healthcare providers. According to an embodiment, the healthcare providers may offer alternative locations, dates, and/or times at which to have appointments. According to an embodiment, the user is able to designate which healthcare providers are able to view the designated location, date, and/or time.

At step 140, the primary healthcare provider updates the user's profile with the user's patient care history and any medical claims that the user may have. This includes any outstanding bills for medical procedures that the user may have.

At step 145, the application grants remote access to the user' information to the user, the primary healthcare provider, and a third party. According to an embodiment, the third party is an insurance provider. According to an embodiment, the user information includes the user's patient care history, the user's insurance company information, the user's billing information, the user's employment information, and any other relevant information.

Granting access to all of these parties aids in the formulation of future patient care and payment of said care by decreasing the amount of time needed to transfer this information to all necessary parties. The granting of this information also enables insurance eligibility to be determined on-demand.

At step 150, communication, using the application, is enabled between the user, the primary healthcare provider, and the third party. This communication may include communication via e-mail, text message, video communication, and audio communication. The communication may also include the transferring of documents, pictures, x-rays, or any other suitable materials. According to an embodiment, this communication enables the creation of centralized health record and centralized bill payments. As described herein, the centralized health record may be stored in a centralized information store of “vault” such that the patient/user, healthcare provider, and third-party insurer may access the vault remotely without use of a physical swipe card. The vault houses the pre-visit identifying documentation for the user and allows for reusability of the information. For example, once the patient uploads identification documentation (e.g., a driver's license, a passport, a health insurance card, etc.) such information remains in the vault.

At step 155, one or more outstanding bills are paid using any payment methods provided by any of the parties. This bills may include payment of any of the parties toward any of the other parties.

It is to be noted that the preceding and following methods and steps may also be used in any field in which multiple participants and/or customers are involved. For example, any of the patients and/or healthcare professionals described above may be customers and/or business representatives and the field may be any field between any of the parties, such as, e.g., the selling of goods, the transferring of information, etc. According to an embodiment, the patient in the preceding and following methods and steps may instead be a consumer. According to an embodiment, the healthcare provider in the preceding and following methods and steps may instead be a seller of goods and/or services. The third party may be any third party relevant to the other parties.

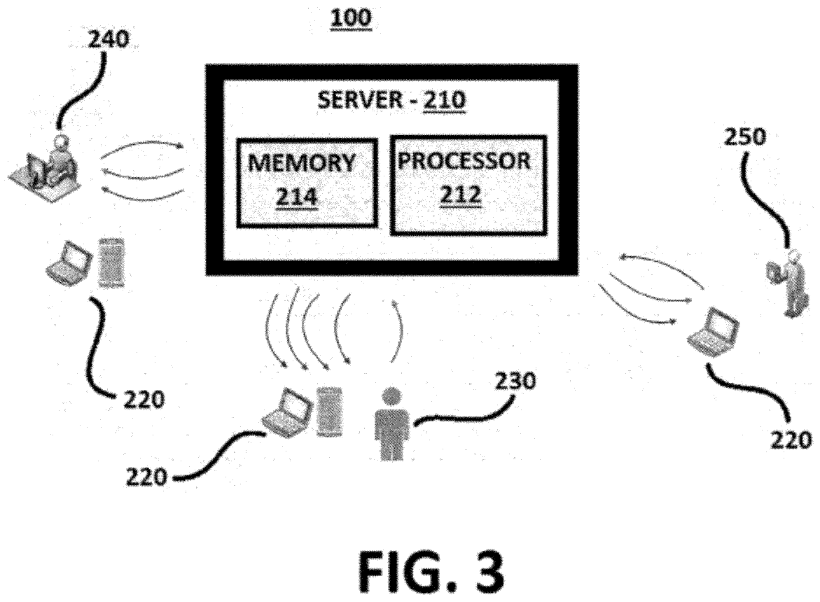

Referring now to FIG. 3, a block/flow diagram of a system 200 for managing healthcare information is illustratively depicted, in accordance with an embodiment of the present invention.

According to an embodiment, the system 200 includes a server 210, which includes at least one processor 212 and memory 214, connected to one or more computing devices 220, which enable a user (patient) 230, a healthcare provider 240, and a third party player 250 (such as, e.g., an insurance company and/or a medical insurance provider) to interact with the server and with each other. The mobile devices may include, but are not limited to, a desktop computer, a laptop computer, a tablet computer, a mobile phone, etc.

According to an embodiment, the processor is configured to run a data retrieval computer application configured to perform one or more of the steps of the method shown in FIGS. 1-2. According to an embodiment, memory 214 is configured to store a database of a plurality of healthcare providers 240, a database of one or more users 230, descriptive information related to each of the healthcare providers 240 and each of users 230, and billing and insurance information.

According to an embodiment of the present invention, the server 210 is connected to a plurality of computing devices 220. Each of these computing devices has access to the data retrieval computer application. This connection, using the data retrieval computer application, enables each of the users (patients) 230, healthcare providers 240, and third party players 250 (such as insurance companies) to interact with the server 110 and with each other through the server 110.

According to an embodiment, the server 200 logs any search parameters used by the user 230, logs/tracks which healthcare providers 240 are being viewed by the user, and/or what information is being viewed for each of the healthcare providers 240.

According to an embodiment, the users 230 are able to select any healthcare providers 240 that they wish to view at a later time, and the memory 214 stores this information so that the users 230 can retrieve it.

According to an embodiment, the healthcare providers 240 include physicians, hospitals, pharmacies, urgent care offices, outpatient service centers, laboratories, medical transport services, home medical equipment (HME)/Durable Medical Equipment (DME) distributors, etc.

According to an embodiment, the system 200 includes an automatic messaging scheduler bot service, which includes a scheduler bot assistant. According to an embodiment, the automatic scheduler bot assistant monitors a service provider's calendar and incoming appointment request message queue. In the event that a service provider receives a request for an appointment for a given date and time slot, a “Scheduler Bot” will verify the availability of the service provider for the requested time slot, automatically place a hold on the service provider's calendar for the time slot, and send a tentative confirmation for the appointment to the requestor for the acceptance of the appointment. If the appointment requestor accepts the appointment confirmation sent by the Scheduler Bot within a defined period of time, the Scheduler Bot will confirm the appointment on the service provider's calendar. Otherwise, it will expire the hold placed on the service provider's calendar.

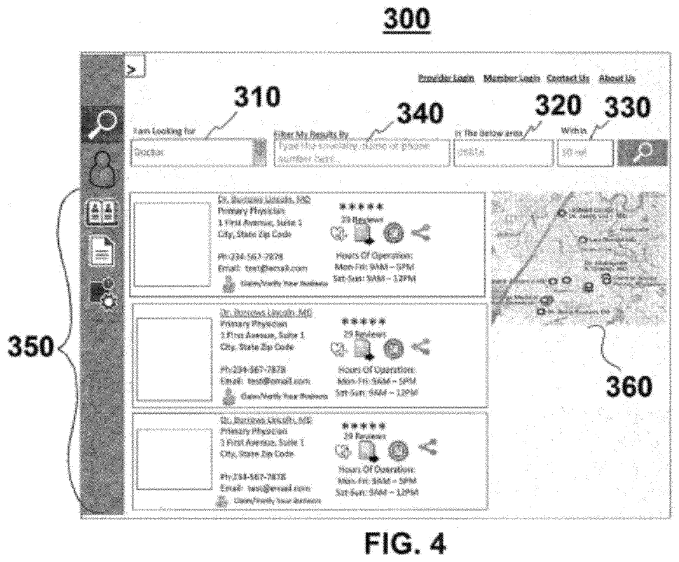

Referring now to FIG. 4, a screenshot of a healthcare provider search engine 300 of the data retrieval application is illustratively shown, in accordance with an embodiment of the present invention.

According to an embodiment, the search engine 300 includes multiple search parameters with which a user can narrow a search for a healthcare provider. According to the embodiment shown in FIG. 4, the search parameters include the type 310 of healthcare provider that the user is wishing to find, the geographical location 320 of the healthcare provider, and the distance 330 within that area. According to the embodiment shown in FIG. 4, the healthcare providers can also be filtered by the type of specialty of the healthcare provider, the name of the healthcare provider, or the phone number of the healthcare provider (collectively 340).

According to an embodiment, any results 350 are shown to the user, which include descriptive information regarding the healthcare providers. According to an embodiment, a map 360 is also presented to the user, enabling the users to locate the healthcare providers on the maps and to acquire directions to one or more of the healthcare providers in the results.

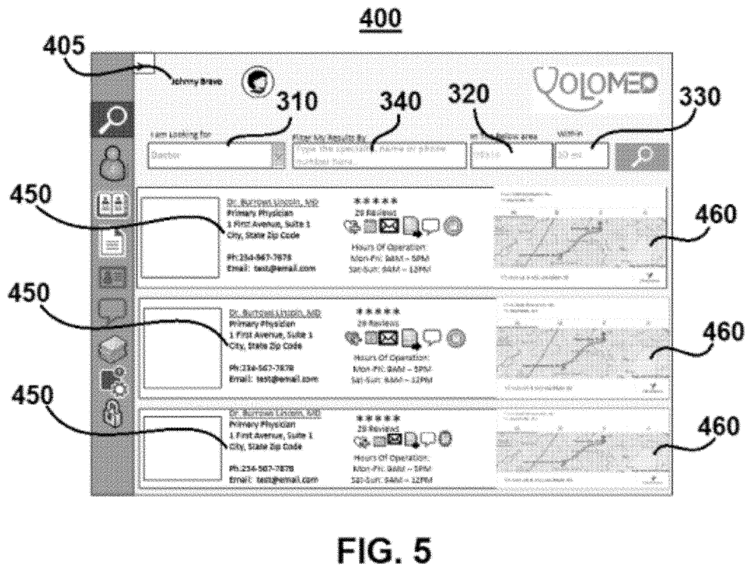

Referring now to FIG. 5, a screenshot of a healthcare provider search engine 400 of the data retrieval application is illustratively shown, in accordance with an embodiment of the present invention. According to an embodiment, the healthcare provider search engine 400 of FIG. 5 is an alternate embodiment of the functionality shown in FIG. 4.

According to an embodiment, the search engine 400 includes a name 405 of a user and multiple search parameters with which the user can narrow a search for a healthcare provider. According to the embodiment shown in FIG. 5, the search parameters include the type 310 of healthcare provider that the user is wishing to find, the geographical location 320 of the healthcare provider, and the distance 330 within that area. According to the embodiment shown in FIG. 4, the healthcare providers can also be filtered by the type of specialty of the healthcare provider, the name of the healthcare provider, or the phone number of the healthcare provider (collectively 340).

According to an embodiment, any results 450 are shown to the user, which include descriptive information regarding the healthcare providers. According to an embodiment, each of the medical professionals 450 returned in the results includes a map 460 indicating the user's distance to each of the medical professionals 450 and also enabling the users to locate the healthcare providers 450 on the maps.

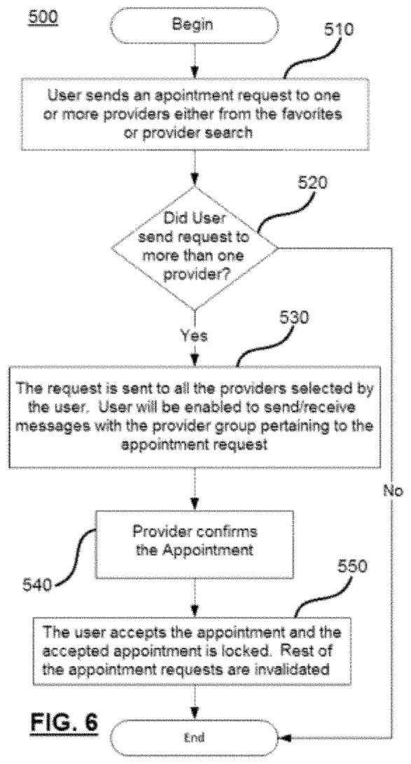

Referring now to FIG. 6, a flowchart of a method 500 for scheduling a medical appointment is illustratively depicted, in accordance with an embodiment of the present invention.

At step 510, a user sends an appointment request to one or more healthcare providers. According to an embodiment, the request indicates the time of the requested appointment, the subject matter of the requested appointment, and/or any other relevant information related to the requested appointment such as, but not limited to, the name of the user, the age of the user, the medical history of the user, etc. According to an embodiment, the healthcare providers are accessed from the user's favorites list and/or from the results of a healthcare provider search. According to an embodiment, the user is able to send a request to more than one healthcare provider.

At step 520, it is determined whether the user sent a request to more than one healthcare provider. If the user has not sent a request to a healthcare provider, method ends. If the user has sent a request to more than one healthcare provider, the request is sent to all of the healthcare providers selected by the user, at step 530. According to an embodiment, the user is able to send and/or receive message to and/or from the group of healthcare providers pertaining to the requested appointment. According to another embodiment, the user is able to send and/or receive messages to and/or from one or more of the healthcare providers in the group of healthcare providers pertaining to the requested appointment.

At step 540, one or more of the healthcare providers from the group of healthcare providers pertaining to the requested appointment confirms the appointment. At step 550, the user accepts one of the confirmed appointments. After acceptance of the appointment, any remaining appointment requests are invalidated.

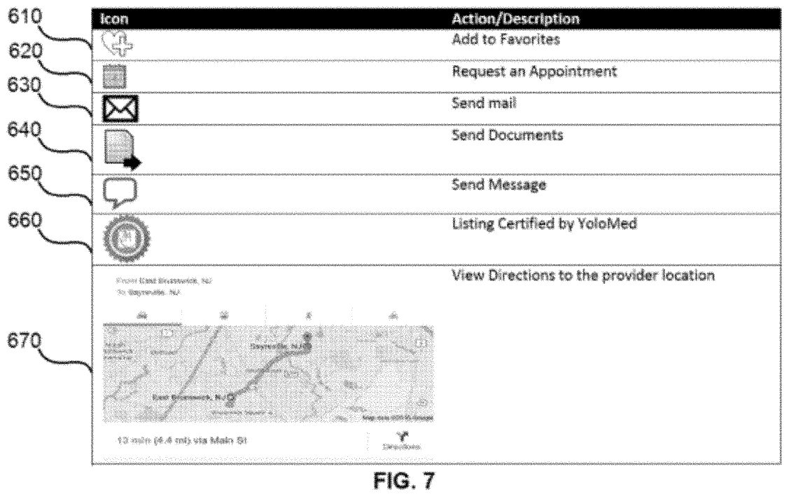

Referring now to FIG. 7, a chart defining various icons used in conjunction with the healthcare provider search engine is illustratively depicted, in accordance with an embodiment of the present invention.

According to an embodiment, the healthcare provider search engine may include, e.g., an icon 610 indicating that a healthcare provider will be added to the user's favorites list, an icon 620 enabling a user to request an appointment, an icon 630 enabling a user to send mail, an icon 640 enabling a user to send one or more documents, an icon 650 enabling a user to send a message, an icon 660 indicating that a listing is a certified listing, and an icon 670 indicating a map with directions to a location of one or more healthcare providers. It is noted that other suitable icons may also be used while maintaining the spirit of the present invention.

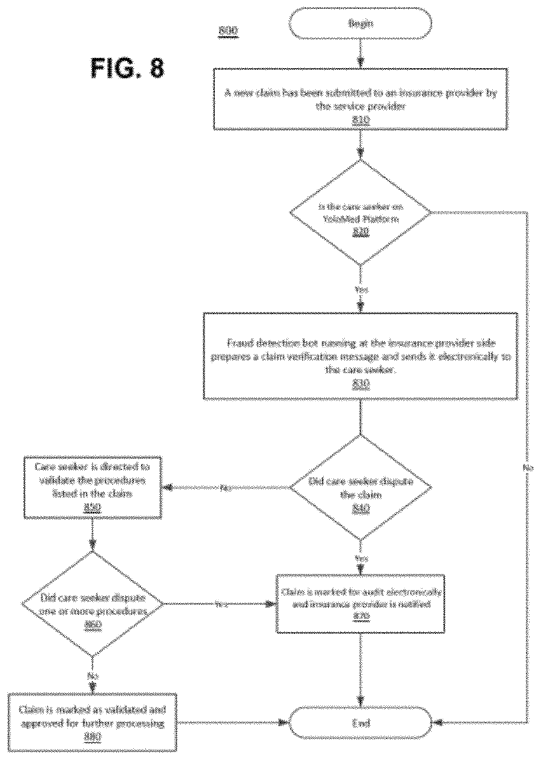

Referring now to FIG. 8, a flowchart of a method 800 of using a fraud detection bot is illustratively depicted, in accordance with an embodiment of the present invention. The “Fraud Detection Bot” provided by YoloMed facilitates the fast electronic validation of claims being processed by Insurance providers via CMS, EDI etc. by sending a review form to the entity requesting service. Currently there is no easy way for insurance provider to communicate with a service seeker in the event of a claim being submitted on the care seeker's behalf. The Fraud Detection Bot monitors new claims being submitted to an insurance provider (at step 810) and automatically prepares and sends a claim verification message (at step 830) to the care seeker for validation electronically. The claim verification message may include a textual message, an audio message, and/or a graphical message. In examples, the claim verification message may be an email, a text message, an audio message, etc.

According to an embodiment, it is first determined, at step 820, whether the care seeker is on the YoloMed platform. Once the message is sent to the care seeker, the care seeker will have an option to confirm or dispute (at step 840) the entire claim or any line items on the claim. If the care seeker disputes claim or any line items on the claim, the entire claim or any line items on the claim will be marked electronically/flagged for further audit. The insurance provider is also notified of such flagging (at step 870).

If the care seeker does not dispute the claim or any line items on the claim, the care seeker is directed to validate the procedures listed in the claim (at step 850). The care seeker then has the option to confirm or dispute (at step 860) one or more of the procedures. If the care seeker does not dispute one or more of the procedures, then, at step 880, the claim is marked as validated and approved for further processing. If the care seeker does dispute one or more procedures, the claim will be marked electronically for further audit and insurance provider is notified (at step 870).

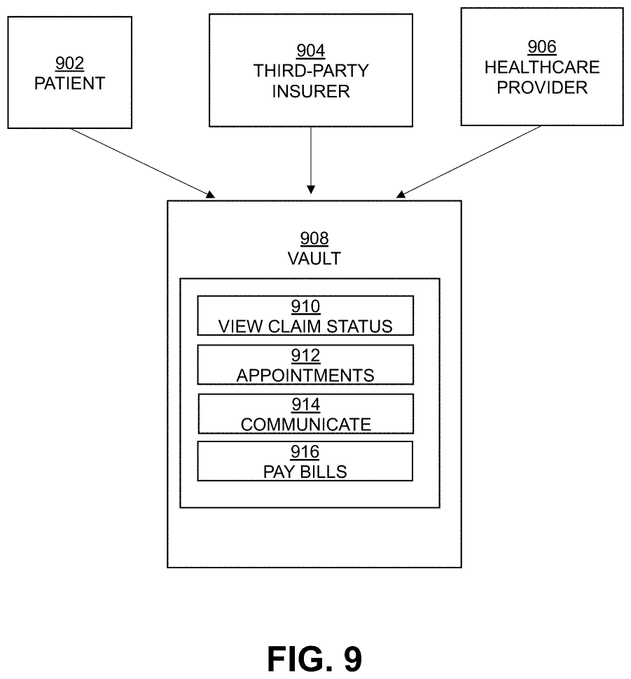

FIG. 9 shows a block diagram of communication with a centralized information store or vault, according to an embodiment of the present invention.

As shown in FIG. 9, a patient 902 checks in at a kiosk physically located at a healthcare providers office or online via the data retrieval computer application. Such provides the patient 902 with access to the vault (e.g., the centralized information store) 908, where the patient 902 can make healthcare appointments 912, view claim status 910, pay healthcare bills 916, and/or communicate 914 with the healthcare provider 906 and/or the third-party insurer 904. The healthcare provider 906 and/or the third-party insurer 904 are also able to access the vault 908 to view the claim status 910 of the patient 902 and/or communicate 914 with one another. It should be appreciated that the vault 908 may be accessed via the data retrieval application on a mobile device and/or the web.

The patient 902 is also capable of accessing other modules via the data retrieval application on the mobile device and/or the web. In one example, the patient 902 accesses a module that interacts with a healthcare provider's practice via JSON and an HL7 interface. As described herein, “JSON” is an open standard file format, and data interchange format, that uses human-readable text to store and transmit data objects consisting of attribute-value pairs and array data types. As described herein “Health Level Seven” or “HL7” is a set of international standards used to provide guidance with transferring and sharing data between various healthcare providers. The “HL7 interface” is configured so that one system serves as a demographic master and conserves all patient information, which can flow automatically into another system. This configuration dismisses the need to key data into multiple systems. Such configuration between the module and the healthcare provider's practice allows for registration of patients, updating patient information, updating patient payment information, etc. Furthermore, the module includes access to resources (e.g., physicians, administrative staff, etc.), patient information (e.g., demographics, insurance information, notes, visit history, notifications, etc.), and appointments.

The patient 902 may also access another module via the data retrieval application on the mobile device and/or the web that provides services, such as email services, fax services, interactive voice response (IVR) services, SMS services, etc. This other module provides for telephone reminders regarding appointments and payments. The email service allows for the transfer of documentation, such as Health Insurance Portability and Accountability Act (HIPAA) forms, a provider information form (PIF), an assignment of benefit (AOB) form, an advance beneficiary notice (ABN) form, a patient consent form, etc.

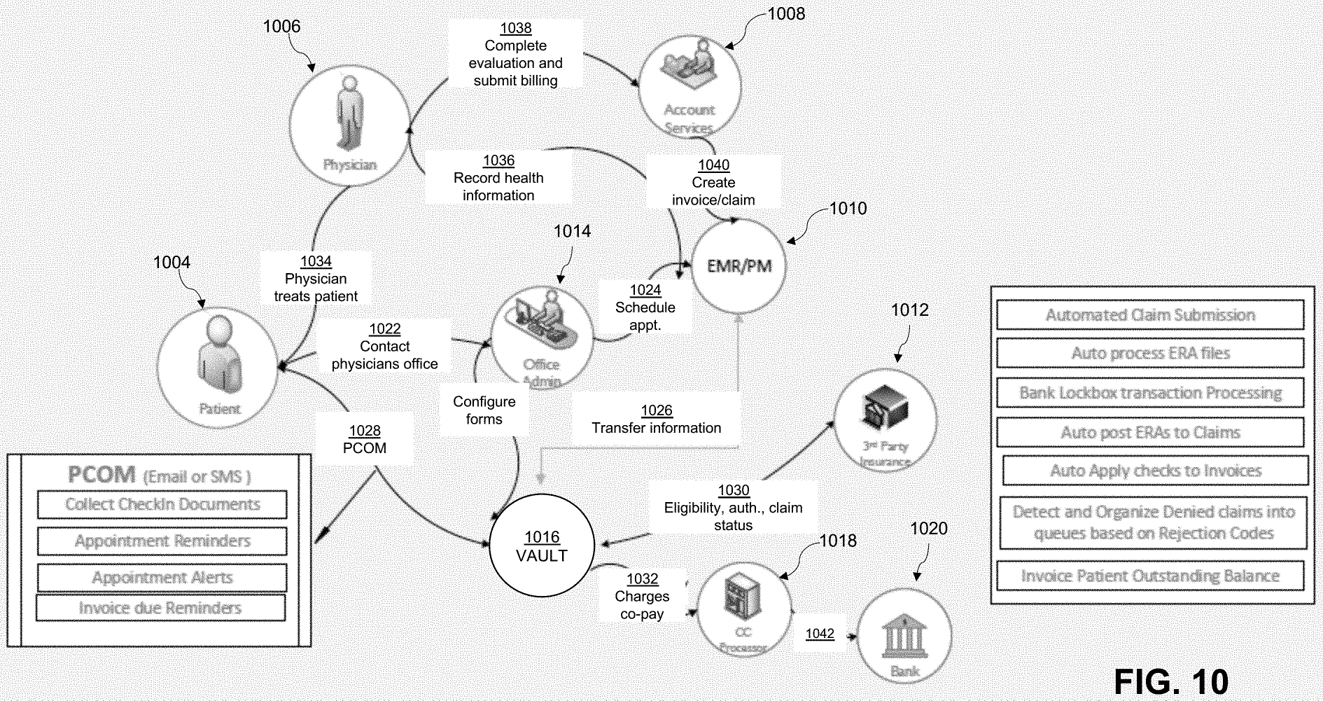

FIG. 10 shows a schematic diagram of data flow between a patient, a healthcare provider, and a third-party insurer in a system for managing healthcare information, according to an embodiment of the present invention.

As shown in FIG. 10, a patient 1004 first contacts the healthcare providers office for an appointment in a process step 1022. The office administrative staff 1014 at the healthcare providers office schedules the appointment in a process step 1024. Such appointment and various patient information is saved via electronic medical records (EMRs) and/or practice management (PM) software 1010. As described herein, “EMRs” are a digital version of the paper charts in the clinician's office. An EMR contains the medical and treatment history of the patients in one practice. As described herein, “PM” software is used to collect patient demographics, patient insurance detail and the healthcare services and related diagnoses provided. Next, in a process step 1026, the patient information and appointment information is transmitted to the vault 1016 described herein.

Furthermore, the patient 1004 may receive patient electronic communication (PCOM) 1028 from the system described herein, which may occur via SMS or email message and may include: appointment reminders, appointment alerts, and/or invoice reminders, etc. Moreover, once the patient 1004 is treated by the healthcare provider (e.g., the physician 1006) in a process step 1034, the health information is recorded in a process step 1036 and such information is updated in the vault 1016. Once the healthcare provider (e.g., the physician 1006) treats the patient 1004, the physician 1006 transmits the patient evaluation and billing information to account services 1008 in a process step 1038. Account services 1008 then creates an invoice or claim for the appointment in a process step 1040. A third-party insurer 1012 may access the vault 1016 to view/review patient claim status and authorization information, among other types of information, in a process step 1030. The patient 1004 may then be charged a co-pay for the appointment at the healthcare providers office in a process step 1032. Next, a credit card processor 1018 receives the payment 1042 from a bank 1020.

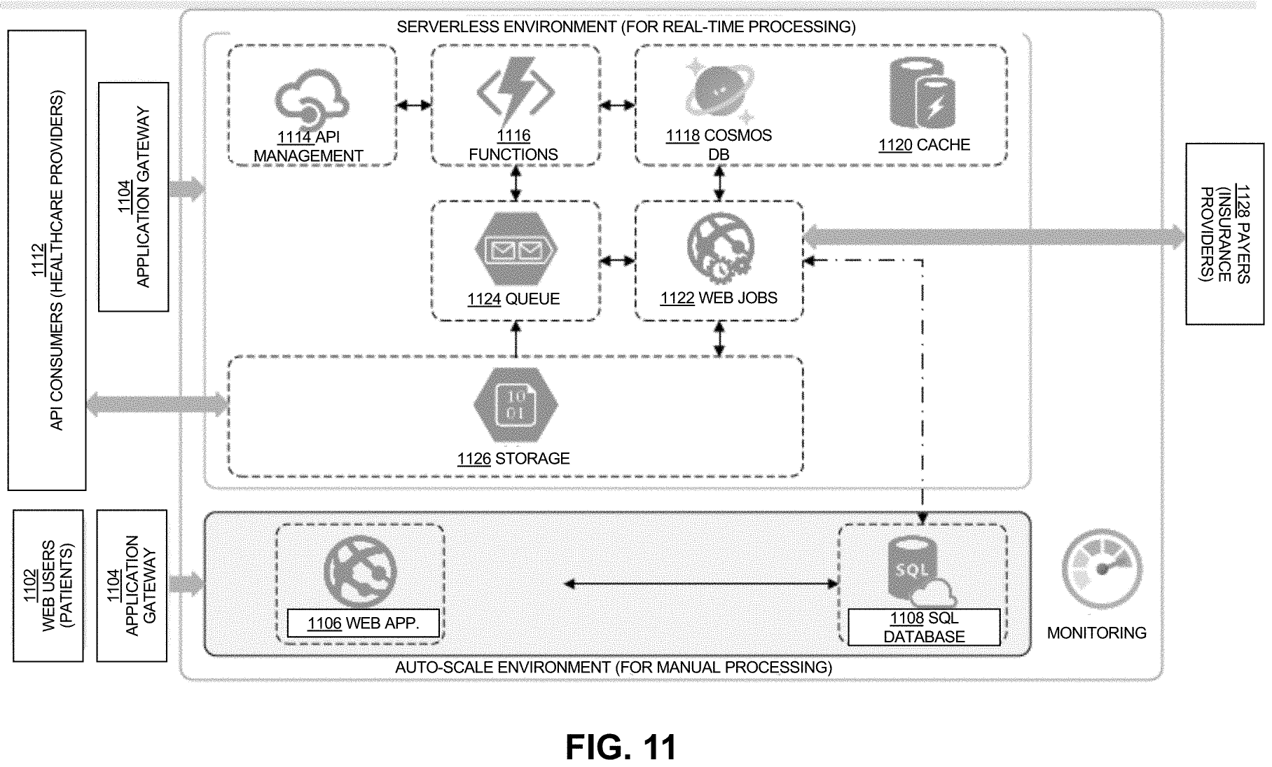

FIG. 11 shows a schematic diagram of cloud architecture of a system for managing healthcare information, according to an embodiment of the present invention.

As shown in FIG. 11, the cloud architecture of the system includes an auto-scale environment and a serverless environment. The auto-scale environment may be used for manual processing of information and the serverless environment may be used for real-time processing of information. Web-users/patients 1102 may utilize an application gateway 1104 to access a web application 1106 of the auto-scale environment. The web application 1106 may interact with an SQL database 1108 of the auto-scale environment.

API consumers (e.g., healthcare providers) 1112 may utilize the application gateway 1104 to access the serverless environment. The serverless environment may include numerous modules or engines, such as a storage engine 1126, an API management engine 1114, a functions engine 1116, a queue engine 1124, and/or a web jobs engine 1122, among others. The serverless environment may also include a cosmos database 1118 and/or a cache 1120. The SQL database 1108 and/or payers (e.g., insurance providers) 1128 may interact with one or more engines of the serverless environment.

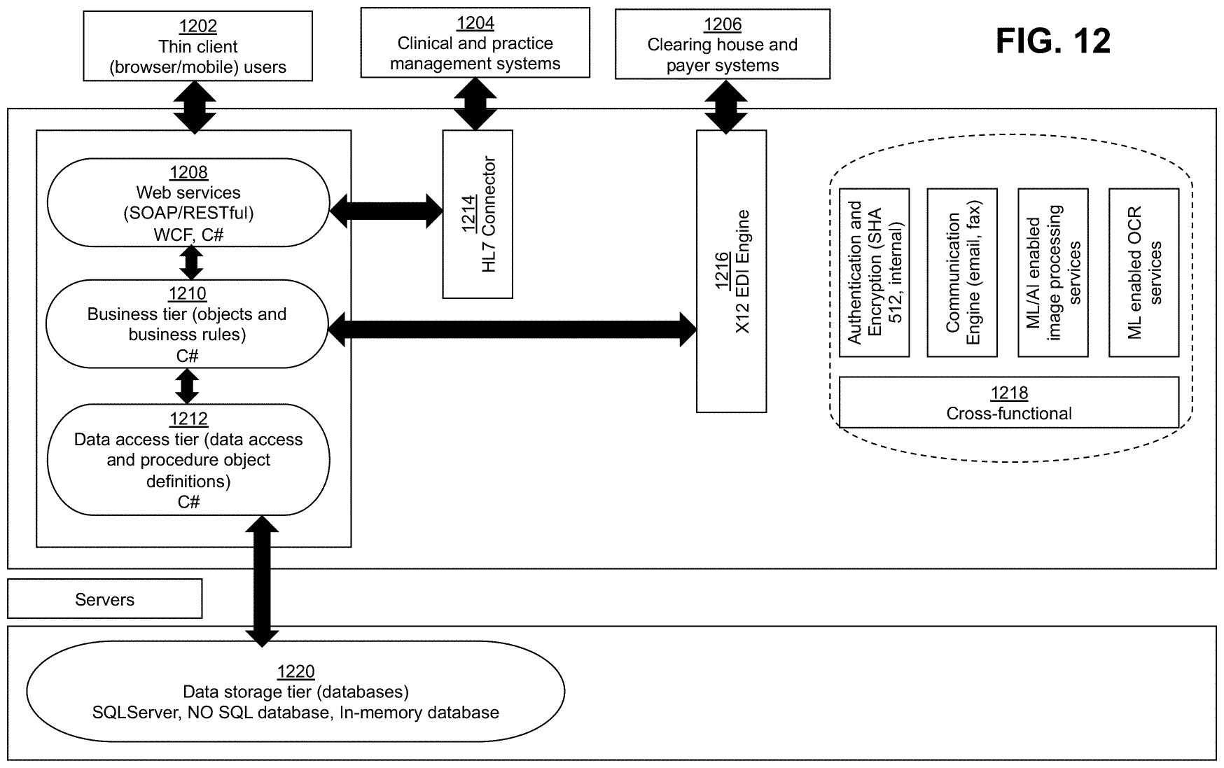

FIG. 12 shows a schematic diagram of architecture/technology stack of a system for managing healthcare information, according to an embodiment of the present invention.

The architecture/technology stack of the system for managing healthcare information of FIG. 12 includes thin client users 1202, clinical and practice management systems 1204, and clearing house and payer systems 1206. The thin client users 1202 may interact with web services 1208. The web services 1208 may interact via an HL7 connector 1214 with the clinical and practice management systems 1204. Moreover, the web services 1208 may interact with a business tier 1210, which may interact with a data access tier 1212.

The business tier 1210 may interact with the clearing house and payer systems 1206 via an X12 EDI engine 1216, or another similar engine. Cross-functionality 1218 may also be provided, which includes: authentication, encryption, communication (e.g., email, fax, etc.), ML/AI-enabled image processing services, and/or ML-enabled optical character recognition (OCR) services. As described herein, “OCR” is the electronic or mechanical conversion of images of typed, handwritten or printed text into machine-encoded text, whether from a scanned document, a photo of a document, a scene-photo or from subtitle text superimposed on an image. Further, the data access tier 1212 may interact with a data storage tier 1220.

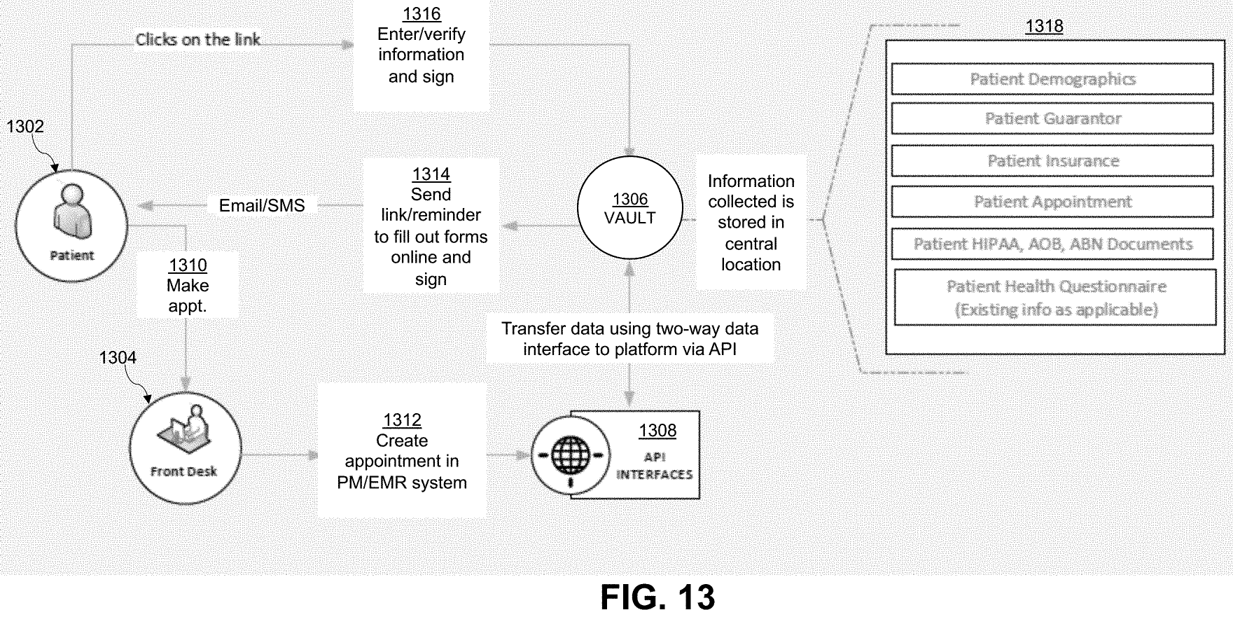

FIG. 13 shows a schematic diagram of patient information and signature capture prior to visiting a healthcare provider, according to an embodiment of the present invention.

As shown in FIG. 13, a patient 1302 may first call a healthcare providers office to make an appointment (e.g., a process step 1310). A front desk 1304 at the healthcare providers office may create the appointment in the PM/EMR system(s) (e.g., a process step 1312). The appointment information and/or patient information may be transferred using a two-way data interface to a platform via an API. API interfaces 1308 may also be used. Such information may be saved in the vault 1306. Other information 1318 stored in the vault may include: patient demographic information, patient guarantor information, patient insurance information, patient appointment information, patient HIPAA documentation, patient AOB documentation, patient ABN documentation, patient healthcare questionnaire, etc. The patient 1302 may then receive, via email or SMS, a link and/or a reminder to fill and sign forms online prior to arriving at the scheduled appointment (e.g., a process step 1314). Once the patient 1302 accesses the link, the patient may enter or verify information and may electronically sign the documents (e.g., a process step 1316).

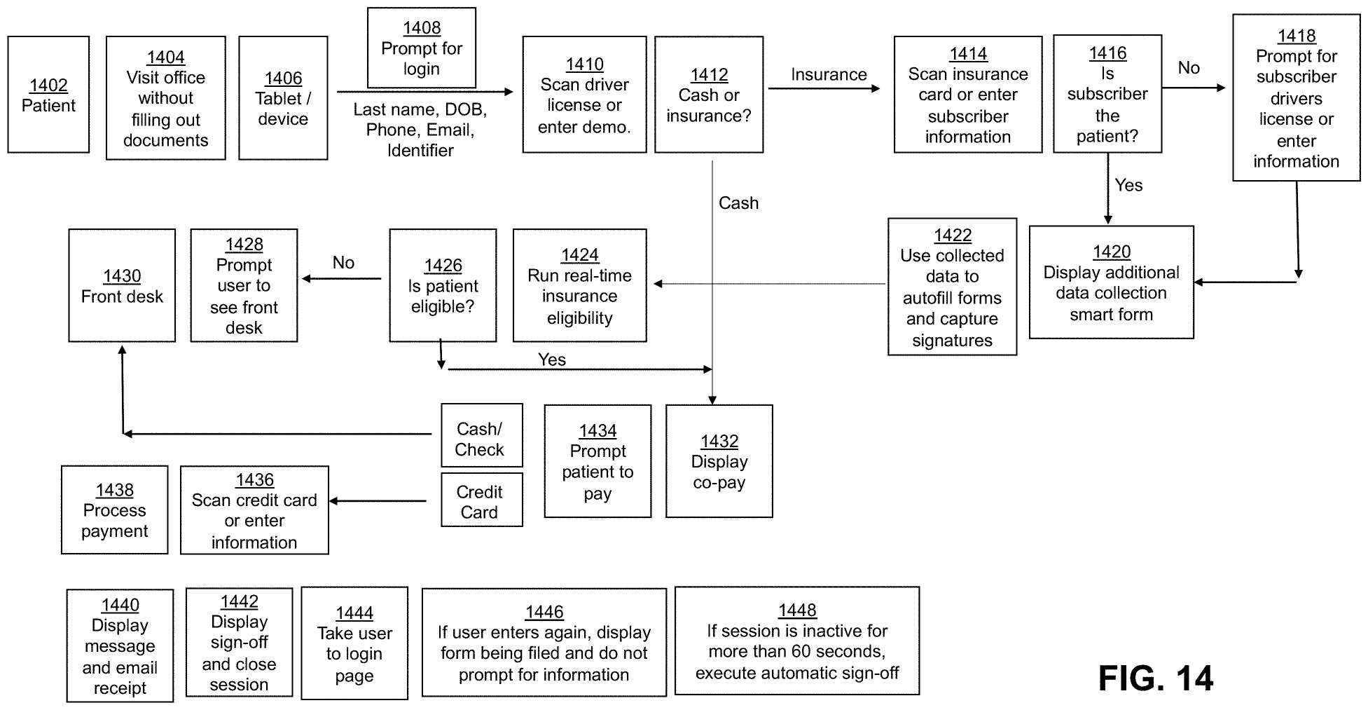

FIG. 14 shows a schematic diagram of patient check-in at a kiosk in a waiting room at a healthcare provider, according to an embodiment of the present invention.

It should be appreciated that the kiosk described herein may be physically located anywhere in the healthcare providers office. As shown in FIG. 14, a user/patient 1402 may visit the healthcare providers office without filling out any forms or documentation (e.g., a process step 1404). The user/patient 1402 may then be prompted, via the kiosk, a tablet, or device 1406, to input information, such as a name, a date of birth, a telephone number, an email address, and/or an identifier (e.g., a process step 1408). Next, the kiosk may prompt the user/patient 1402 to scan the users driver's license via the kiosk/device or to enter demographic information (e.g., a process step 1410). The kiosk may then inquire whether the user/patient 1402 is paying cash for the appointment or will be utilizing healthcare insurance to pay for the appointment (e.g., a process step 1412).

If the user/patient 1402 will be utilizing cash to pay for the appointment, the co-pay and a previous balance (if any) will be displayed to the user/patient 1402 via the kiosk (e.g., a process step 1432). The user/patient 1402 will be prompted to pay the co-pay and the previous balance (if any) via credit card, debit card, cash, or check (e.g., a process step 1434). If the user/patient 1402 is paying via debit card or credit card, the user/patient 1402 may swipe the debit card or the credit card or may manually enter information associated with the debit card or the credit card into the kiosk (e.g., a process step 1436). If the user/patient 1402 is paying via cash or a check, the user/patient 1402 may pay for the appointment at the front desk 1430 of the healthcare providers office.

If the user/patient 1402 is paying via debit card or credit card, the kiosk processes the payment and displays a successful message via the kiosk to the user/patient 1402 (e.g., a process step 1438 and a process step 1440). The user/patient 1402 is then prompted to select to receive the receipt of the transaction via email and/or print (e.g., the process step 1440). The user/patient 1402 may then log-out of the system (e.g., a process step 1442, a process step 1444, and/or a process step 1446). In some instances, if the user/patient 1402 is inactive with the kiosk for more than a predetermined amount of time, the kiosk automatically signs the user/patient 1402 off (e.g., a process step 1448). In some examples, this predetermined amount of time is 60 seconds. However, the predetermined amount of time is not limited to any particular time or range of time.

If the user/patient 1402 will be utilizing medical insurance to pay for the appointment, the kiosk prompts the user/patient 1402 to scan the health insurance card or to input the health insurance information into the kiosk (e.g., a process step 1414). The kiosk then determines if the subscriber to the health insurance is the user/patient 1402 (e.g., a process step 1416). If the subscriber to the health insurance is the user, the kiosk displays an additional data collection smart form created by the healthcare provider and uses collected data to autofill the form (e.g., a process step 1420). If the subscriber to the health insurance is not the user, the kiosk prompts the user to scan the subscribers driver's license or to enter various demographic information for the subscriber (e.g., a process step 1418). Then, the kiosk displays an additional data collection smart form created by the healthcare provider and uses collected data to autofill the form (e.g., the process step 1420).

An electronic signature of the user is then captured by the kiosk (e.g., a process step 1422). Then, the system runs real-time insurance eligibility for the user (e.g., a process step 1424). If the user is eligible (determined at a process step 1426), the co-pay is displayed (e.g., a process step 1432) and the user proceeds through the process to pay for the appointment. If the user is not eligible, the user is prompted to visit the front desk of the healthcare providers office for further assistance (e.g., a process step 1428).

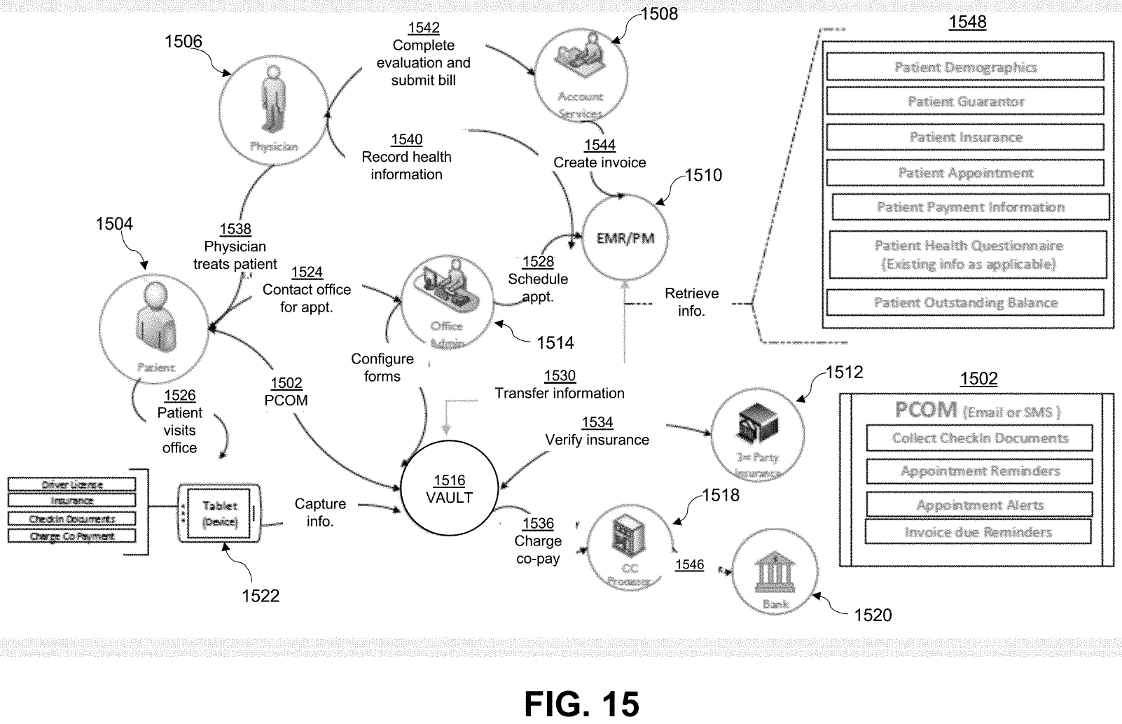

FIG. 15 shows a schematic diagram of a kiosk implementation in a waiting room at a healthcare provider, according to an embodiment of the present invention. It should be appreciated that the kiosk implementation is substantially similar to the schematic diagram of data flow between the patient, the healthcare provider, and the third-party insurer in FIG. 10. As shown in FIG. 15, a patient 1504 first contacts the healthcare providers office for an appointment in a process step 1524. The office administrative staff 1514 at the healthcare providers office schedules the appointment in a process step 1528. Moreover, appointment information and various patient information is saved via electronic medical records (EMRs) and/or practice management (PM) software 1510. Next, in a process step 1530, the patient information and appointment information is transmitted to the vault 1516 described herein.

When the patient 1504 visits the healthcare providers office (e.g., a process step 1526), the patient 1504 may be prompted to utilize the kiosk/device 1522 to scan or input information from a drivers license, insurance card, or other information. Such information is also saved in the vault 1516.

Furthermore, the patient 1504 may receive patient electronic communication (PCOM) 1502 from the system described herein, which may occur via SMS or email message and may include: appointment reminders, appointment alerts, and/or invoice reminders, etc.

Moreover, once the patient 1504 is treated by the healthcare provider (e.g., the physician 1506) in a process step 1538, the health information is recorded in a process step 1540 and such information is updated in the vault 1516. Other information 1548 that may be stored in the vault 1516 includes: patient demographics, patient guarantor, patient insurance, patient appointment information, patient payment information, patient healthcare questionnaire, patient outstanding balance, etc. Once the healthcare provider (e.g., the physician 1506) treats the patient 1504, the physician 1506 transmits the patient evaluation and billing information to account services 1508 in a process step 1542.

Account services 1508 then creates an invoice or claim for the appointment in a process step 1544. A third-party insurer 1512 may access the vault 1516 to verify insurance and/or view/review patient claim status and authorization information, among other types of information, in a process step 1534. The patient 1504 may then be charged a co-pay for the appointment at the healthcare providers office in a process step 1536. Next, a credit card processor 1518 receives the payment 1546 from a bank 1520.

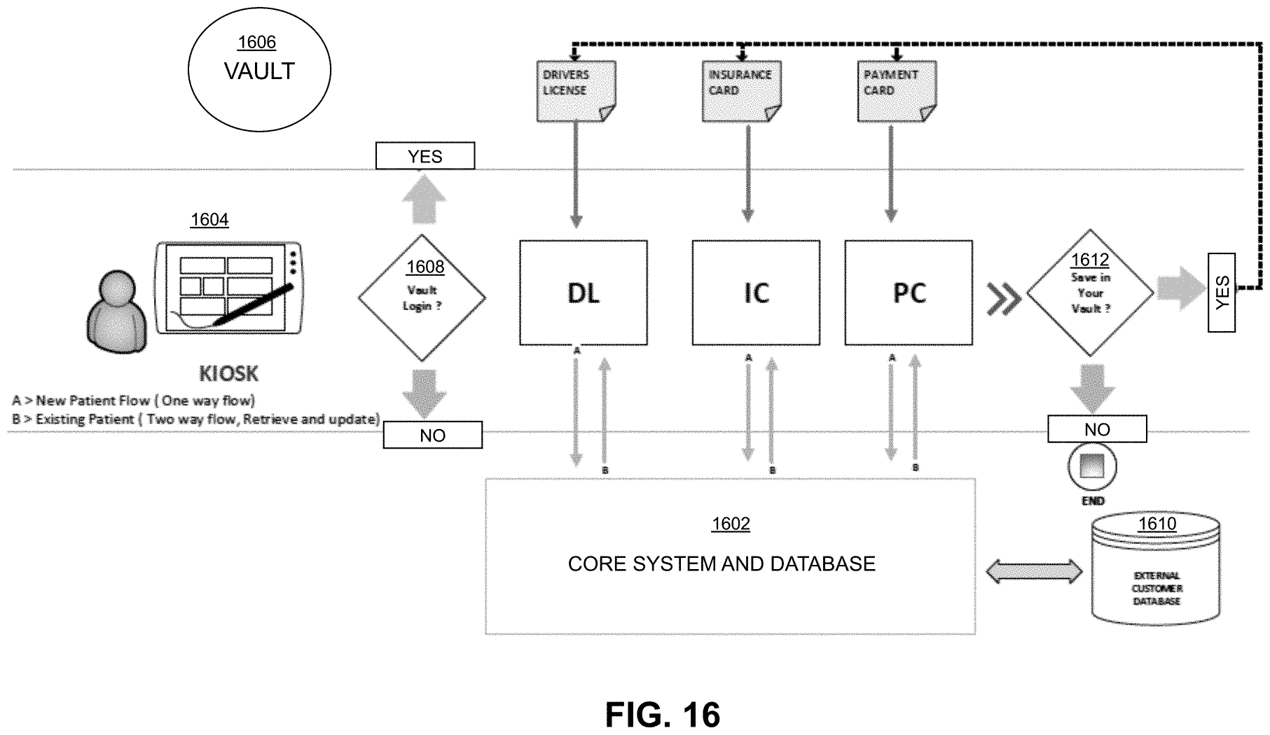

FIG. 16 shows a schematic diagram of kiosk data flow and integration with a centralized information store or vault, according to an embodiment of the present invention.

As shown in FIG. 16, the core system and database 1602 is configured to interact with an external customer database 1610. The core system and database 1602 and the vault 1606 both interact with the kiosk 1604. At the kiosk 1604, if the user is an existing patient, there is two-way data flow to the vault such that the patient data is retrieved and updated. If the user at the kiosk 1604 is a new patient, there is one-way data flow to the core system and database 1602.

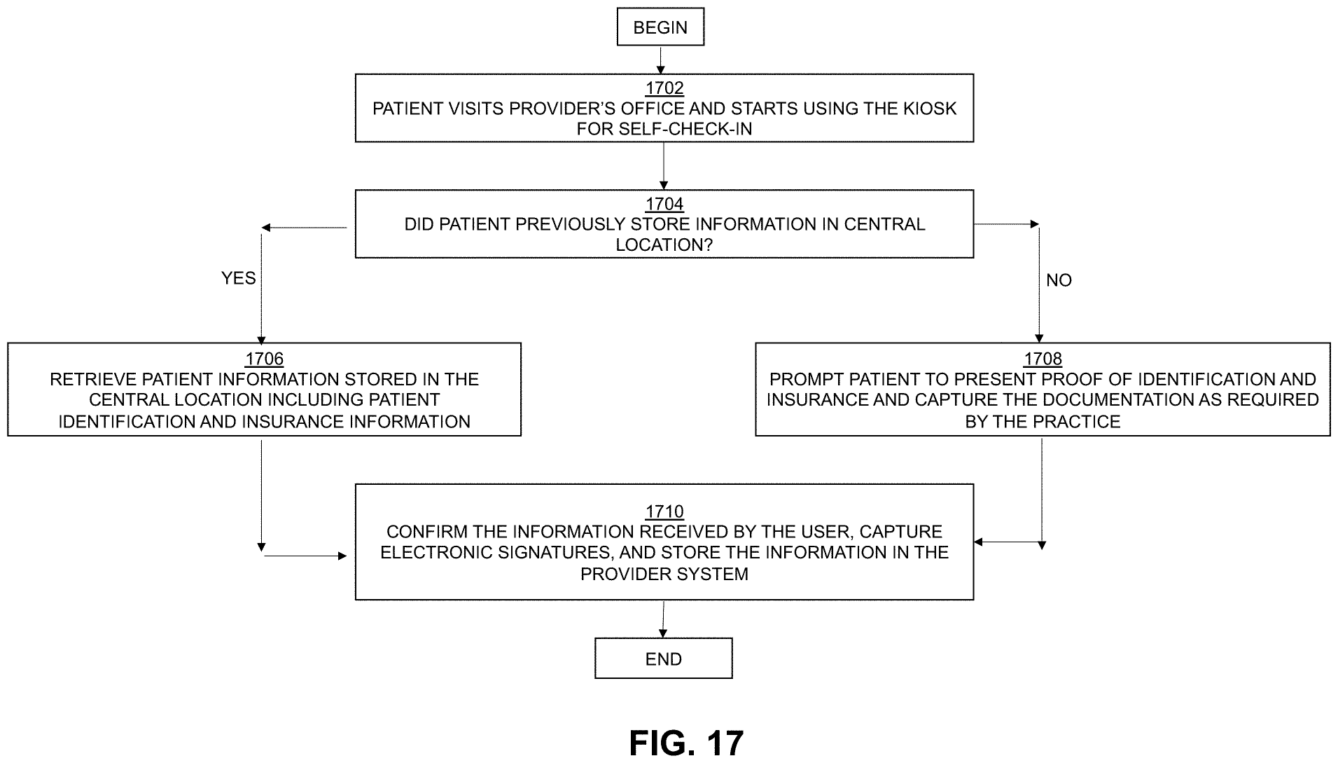

FIG. 17 depicts a flowchart of a method for reusing information across multiple providers from a kiosk at a health provider, according to an embodiment of the present invention.

As shown in FIG. 17, the method for reusing information across multiple providers from a kiosk at a health provider begins with a process step 1702 that includes the patient visiting the healthcare providers office and beginning the self-check-in process using the kiosk. A process step 1704 follows the process step 1702 and includes determining if the patient has previously stored information in the central location (e.g., the vault). If the answer to the process step 1704 is YES, the method proceeds to a process step 1706. If the answer to the process step 1704 is NO, the method proceeds to a process step 1708.

The process step 1706 includes retrieving the patient information stored in the vault, which includes the patient identification and insurance information, among other information. The process step 1708 includes prompting the patient to present proof of identification and insurance such that the kiosk can capture such documentation.

Next, a process step 1710 follows the process step 1706 or the process step 1708. The process step 1710 includes the kiosk confirming the information received by the patient/user, capturing electronic signatures of the patient, and storing the information in the provider system. The process step 1710 ends the method of FIG. 17.

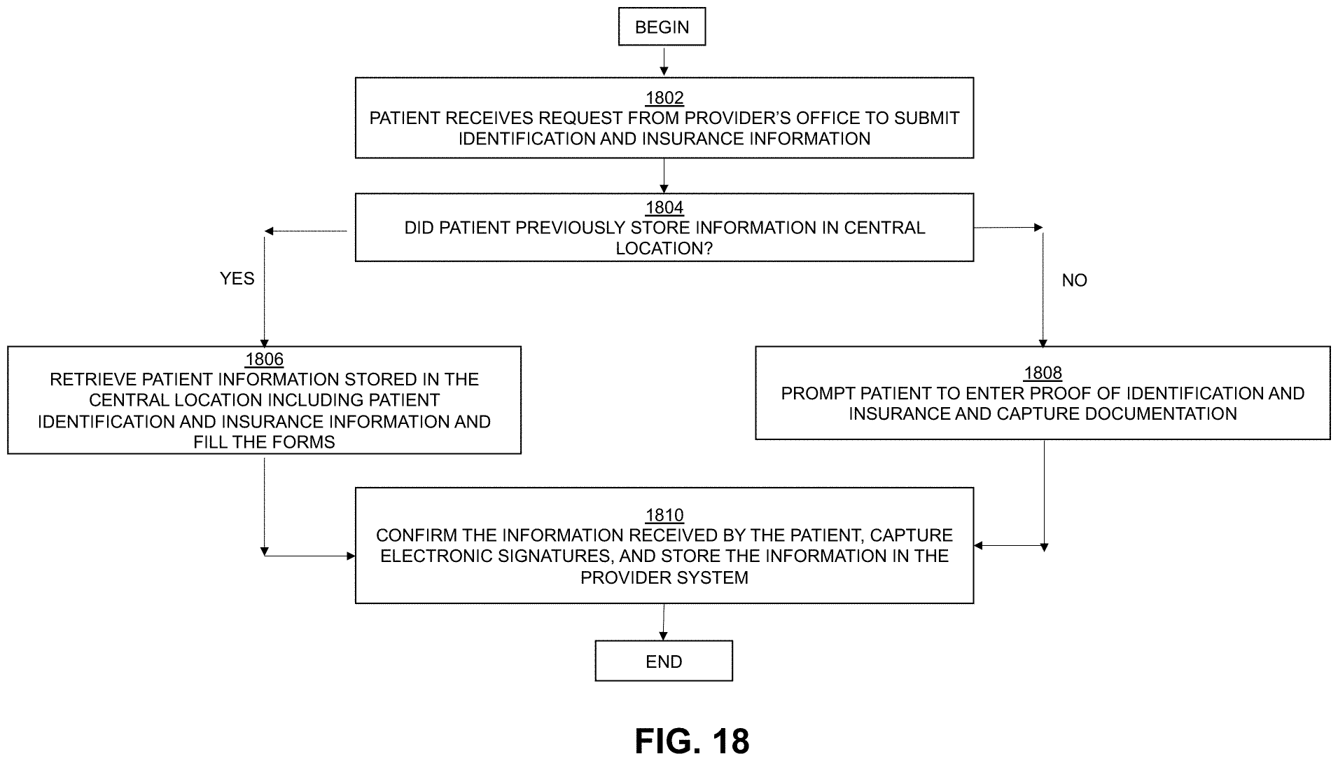

FIG. 18 depicts a flowchart of a method for reusing information across multiple providers from an application executed on a mobile device, according to an embodiment of the present invention.

As shown in FIG. 18, the method for reusing information across multiple providers from an application executed on a mobile device begins at a process step 1802 that includes the patient receiving a request via the mobile device from the healthcare providers office to submit patient identification and insurance information. A process step 1804 follows the process step 1802 and includes determining if the patient has previously stored information in the central location (e.g., the vault). If the answer to the process step 1804 is YES, the method proceeds to a process step 1806. If the answer to the process step 1804 is NO, the method proceeds to a process step 1808.

The process step 1806 includes retrieving the patient information stored in the vault, which includes the patient identification and insurance information, among other information. The process step 1806 further includes automatically populating several forms. The process step 1808 includes prompting the patient to input proof of identification and insurance such that the information may be captured.

Next, a process step 1810 follows the process step 1806 or the process step 1808. The process step 1810 includes confirming the information received by the patient/user, capturing electronic signatures of the patient, and storing the information in the provider system. The process step 1810 ends the method of FIG. 18.

### Systems, Devices and Operating Systems

Typically, a user or users, which may be people or groups of users and/or other systems, may engage information technology systems (e.g., computers) to facilitate operation of the system and information processing. In turn, computers employ processors to process information and such processors may be referred to as central processing units (CPU). One form of processor is referred to as a microprocessor. CPUs use communicative circuits to pass binary encoded signals acting as instructions to enable various operations. These instructions may be operational and/or data instructions containing and/or referencing other instructions and data in various processor accessible and operable areas of memory (e.g., registers, cache memory, random access memory, etc.). Such communicative instructions may be stored and/or transmitted in batches (e.g., batches of instructions) as programs and/or data components to facilitate desired operations. These stored instruction codes, e.g., programs, may engage the CPU circuit components and other motherboard and/or system components to perform desired operations.

One type of program is a computer operating system, which, may be executed by CPU on a computer; the operating system enables and facilitates users to access and operate computer information technology and resources. Some resources that may be employed in information technology systems include: input and output mechanisms through which data may pass into and out of a computer; memory storage into which data may be saved; and processors by which information may be processed. These information technology systems may be used to collect data for later retrieval, analysis, and manipulation, which may be facilitated through a database program. These information technology systems provide interfaces that allow users to access and operate various system components.

In one embodiment, the present invention may be connected to and/or communicate with entities such as, but not limited to: one or more users from user input devices; peripheral devices; an optional cryptographic processor device; and/or a communications network. For example, the present invention may be connected to and/or communicate with users, operating client device(s), including, but not limited to, personal computer(s), server(s) and/or various mobile device(s) including, but not limited to, cellular telephone(s), smartphone(s) (e.g., iPhone®, Blackberry®, Android OS-based phones etc.), tablet computer(s) (e.g., Apple iPad™ HP Slate™, Motorola Xoom™, etc.), eBook reader(s) (e.g., Amazon Kindle™, Barnes and Noble's Nook™ eReader, etc.), laptop computer(s), notebook(s), netbook(s), gaming console(s) (e.g., XBOX Live™, Nintendo® DS, Sony PlayStation® Portable, etc.), portable scanner(s) and/or the like.

Networks are commonly thought to comprise the interconnection and interoperation of clients, servers, and intermediary nodes in a graph topology. It should be noted that the term “server” as used throughout this application refers generally to a computer, other device, program, or combination thereof that processes and responds to the requests of remote users across a communications network. Servers serve their information to requesting “clients.” The term “client” as used herein refers generally to a computer, program, other device, user and/or combination thereof that is capable of processing and making requests and obtaining and processing any responses from servers across a communications network. A computer, other device, program, or combination thereof that facilitates, processes information and requests, and/or furthers the passage of information from a source user to a destination user is commonly referred to as a “node.” Networks are generally thought to facilitate the transfer of information from source points to destinations. A node specifically tasked with furthering the passage of information from a source to a destination is commonly called a “router.” There are many forms of networks such as Local Area Networks (LANs), Pico networks, Wide Area Networks (WANs), Wireless Networks (WLANs), etc. For example, the Internet is generally accepted as being an interconnection of a multitude of networks whereby remote clients and servers may access and interoperate with one another.

The present invention may be based on computer systems that may comprise, but are not limited to, components such as: a computer systemization connected to memory.

Computer Systemization

A computer systemization may comprise a clock, central processing unit (“CPU(s)” and/or “processor(s)” (these terms are used interchangeable throughout the disclosure unless noted to the contrary)), a memory (e.g., a read only memory (ROM), a random access memory (RAM), etc.), and/or an interface bus, and most frequently, although not necessarily, are all interconnected and/or communicating through a system bus on one or more (mother)board(s) having conductive and/or otherwise transportive circuit pathways through which instructions (e.g., binary encoded signals) may travel to effect communications, operations, storage, etc. Optionally, the computer systemization may be connected to an internal power source; e.g., optionally the power source may be internal. Optionally, a cryptographic processor and/or transceivers (e.g., ICs) may be connected to the system bus. In another embodiment, the cryptographic processor and/or transceivers may be connected as either internal and/or external peripheral devices via the interface bus I/O. In turn, the transceivers may be connected to antenna(s), thereby effectuating wireless transmission and reception of various communication and/or sensor protocols; for example the antenna(s) may connect to: a Texas Instruments WiLink WL1283 transceiver chip (e.g., providing 802.11n, Bluetooth 3.0, FM, global positioning system (GPS) (thereby allowing the controller of the present invention to determine its location)); Broadcom BCM4329FKUBG transceiver chip (e.g., providing 802.11n, Bluetooth 2.1+EDR, FM, etc.); a Broadcom BCM4750IUB8 receiver chip (e.g., GPS); an Infineon Technologies X-Gold 618-PMB9800 (e.g., providing 2G/3G HSDPA/HSUPA communications); and/or the like.

The system clock typically has a crystal oscillator and generates a base signal through the computer systemization's circuit pathways. The clock is typically coupled to the system bus and various clock multipliers that will increase or decrease the base operating frequency for other components interconnected in the computer systemization. The clock and various components in a computer systemization drive signals embodying information throughout the system. Such transmission and reception of instructions embodying information throughout a computer systemization may be commonly referred to as communications. These communicative instructions may further be transmitted, received, and the cause of return and/or reply communications beyond the instant computer systemization to: communications networks, input devices, other computer systemizations, peripheral devices, and/or the like. Of course, any of the above components may be connected directly to one another, connected to the CPU, and/or organized in numerous variations employed as exemplified by various computer systems.

The CPU comprises at least one high-speed data processor adequate to execute program components for executing user and/or system-generated requests. Often, the processors themselves will incorporate various specialized processing units, such as, but not limited to: integrated system (bus) controllers, memory management control units, floating point units, and even specialized processing sub-units like graphics processing units, digital signal processing units, and/or the like. Additionally, processors may include internal fast access addressable memory, and be capable of mapping and addressing memory beyond the processor itself; internal memory may include, but is not limited to: fast registers, various levels of cache memory (e.g., level 1, 2, 3, etc.), RAM, etc. The processor may access this memory through the use of a memory address space that is accessible via instruction address, which the processor can construct and decode allowing it to access a circuit path to a specific memory address space having a memory state. The CPU may be a microprocessor such as: AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s). The CPU interacts with memory through instruction passing through conductive and/or transportive conduits (e.g., (printed) electronic and/or optic circuits) to execute stored instructions (i.e., program code) according to conventional data processing techniques. Such instruction passing facilitates communication within the present invention and beyond through various interfaces. Should processing requirements dictate a greater amount speed and/or capacity, distributed processors (e.g., Distributed embodiments of the present invention), mainframe, multi-core, parallel, and/or super-computer architectures may similarly be employed. Alternatively, should deployment requirements dictate greater portability, smaller Personal Digital Assistants (PDAs) may be employed.

Depending on the particular implementation, features of the present invention may be achieved by implementing a microcontroller such as CAST's R8051XC2 microcontroller; Intel's MCS 51 (i.e., 8051 microcontroller); and/or the like. Also, to implement certain features of the various embodiments, some feature implementations may rely on embedded components, such as: Application-Specific Integrated Circuit (“ASIC”), Digital Signal Processing (“DSP”), Field Programmable Gate Array (“FPGA”), and/or the like embedded technology. For example, any of the component collection (distributed or otherwise) and/or features of the present invention may be implemented via the microprocessor and/or via embedded components; e.g., via ASIC, coprocessor, DSP, FPGA, and/or the like. Alternately, some implementations of the present invention may be implemented with embedded components that are configured and used to achieve a variety of features or signal processing.

Depending on the particular implementation, the embedded components may include software solutions, hardware solutions, and/or some combination of both hardware/software solutions. For example, features of the present invention discussed herein may be achieved through implementing FPGAs, which are a semiconductor devices containing programmable logic components called “logic blocks”, and programmable interconnects, such as the high performance FPGA Virtex series and/or the low cost Spartan series manufactured by Xilinx. Logic blocks and interconnects can be programmed by the customer or designer, after the FPGA is manufactured, to implement any of the features of the present invention. A hierarchy of programmable interconnects allow logic blocks to be interconnected as needed by the system designer/administrator of the present invention, somewhat like a one-chip programmable breadboard. An FPGA's logic blocks can be programmed to perform the function of basic logic gates such as AND, and XOR, or more complex combinational functions such as decoders or simple mathematical functions. In most FPGAs, the logic blocks also include memory elements, which may be simple flip-flops or more complete blocks of memory. In some circumstances, the present invention may be developed on regular FPGAs and then migrated into a fixed version that more resembles ASIC implementations. Alternate or coordinating implementations may migrate features of the controller of the present invention to a final ASIC instead of or in addition to FPGAs. Depending on the implementation all of the aforementioned embedded components and microprocessors may be considered the “CPU” and/or “processor” for the present invention.

Power Source

The power source may be of any standard form for powering small electronic circuit board devices such as the following power cells: alkaline, lithium hydride, lithium ion, lithium polymer, nickel cadmium, solar cells, and/or the like. Other types of AC or DC power sources may be used as well. In the case of solar cells, in one embodiment, the case provides an aperture through which the solar cell may capture photonic energy. The power cell is connected to at least one of the interconnected subsequent components of the present invention thereby providing an electric current to all subsequent components. In one example, the power source is connected to the system bus component. In an alternative embodiment, an outside power source is provided through a connection across the I/O interface. For example, a USB and/or IEEE 1394 connection carries both data and power across the connection and is therefore a suitable source of power.

Interface Adapters

Interface bus(ses) may accept, connect, and/or communicate to a number of interface adapters, conventionally although not necessarily in the form of adapter cards, such as but not limited to: input output interfaces (I/O), storage interfaces, network interfaces, and/or the like. Optionally, cryptographic processor interfaces similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and/or the like.

Storage interfaces may accept, communicate, and/or connect to a number of storage devices such as, but not limited to: storage devices, removable disc devices, and/or the like. Storage interfaces may employ connection protocols such as, but not limited to: (Ultra) (Serial) Advanced Technology Attachment (Packet Interface) ((Ultra) (Serial) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces may accept, communicate, and/or connect to a communications network. Through a communications network, the controller of the present invention is accessible through remote clients (e.g., computers with web browsers) by users. Network interfaces may employ connection protocols such as, but not limited to: direct connect, Ethernet (thick, thin, twisted pair 10/100/1000 Base T, and/or the like), Token Ring, wireless connection such as IEEE 802.11a-x, and/or the like. Should processing requirements dictate a greater amount speed and/or capacity, distributed network controllers (e.g., Distributed embodiments of the present invention), architectures may similarly be employed to pool, load balance, and/or otherwise increase the communicative bandwidth required by the controller of the present invention. A communications network may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface. Further, multiple network interfaces may be used to engage with various communications network types. For example, multiple network interfaces may be employed to allow for the communication over broadcast, multicast, and/or unicast networks.

Input Output interfaces (I/O) may accept, communicate, and/or connect to user input devices, peripheral devices, cryptographic processor devices, and/or the like. I/O may employ connection protocols such as, but not limited to: audio: analog, digital, monaural, RCA, stereo, and/or the like; data: Apple Desktop Bus (ADB), IEEE 1394a-b, serial, universal serial bus (USB); infrared; joystick; keyboard; midi; optical; PC AT; PS/2; parallel; radio; video interface: Apple Desktop Connector (ADC), BNC, coaxial, component, composite, digital, Digital Visual Interface (DVI), high-definition multimedia interface (HDMI), RCA, RF antennae, S-Video, VGA, and/or the like; wireless transceivers: 802.11a/b/g/n/x; Bluetooth; cellular (e.g., code division multiple access (CDMA), high speed packet access (HSPA(+)), high-speed downlink packet access (HSDPA), global system for mobile communications (GSM), long term evolution (LTE), WiMax, etc.); and/or the like. One typical output device may include a video display, which typically comprises a Cathode Ray Tube (CRT) or Liquid Crystal Display (LCD) based monitor with an interface (e.g., DVI circuitry and cable) that accepts signals from a video interface, may be used. The video interface composites information generated by a computer systemization and generates video signals based on the composited information in a video memory frame. Another output device is a television set, which accepts signals from a video interface. Typically, the video interface provides the composited video information through a video connection interface that accepts a video display interface (e.g., an RCA composite video connector accepting an RCA composite video cable; a DVI connector accepting a DVI display cable, etc.).

User input devices often are a type of peripheral device (see below) and may include: card readers, dongles, finger print readers, gloves, graphics tablets, joysticks, keyboards, microphones, mouse (mice), remote controls, retina readers, touch screens (e.g., capacitive, resistive, etc.), trackballs, trackpads, sensors (e.g., accelerometers, ambient light, GPS, gyroscopes, proximity, etc.), styluses, and/or the like.

Peripheral devices may be external, internal and/or part of the controller of the present invention. Peripheral devices may also include, for example, an antenna, audio devices (e.g., line-in, line-out, microphone input, speakers, etc.), cameras (e.g., still, video, webcam, etc.), drive motors, lighting, video monitors and/or the like.

Cryptographic units such as, but not limited to, microcontrollers, processors, interfaces, and/or devices may be attached, and/or communicate with the controller of the present invention. A MC68HC16 microcontroller, manufactured by Motorola Inc., may be used for and/or within cryptographic units. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Equivalent microcontrollers and/or processors may also be used. Other commercially available specialized cryptographic processors include: the Broadcom's CryptoNetX and other Security Processors; nCipher's nShield, SafeNet's Luna PCI (e.g., 7100) series; Semaphore Communications' 40 MHz Roadrunner 184; Sun's Cryptographic Accelerators (e.g., Accelerator 6000 PCIe Board, Accelerator 500 Daughtercard); Via Nano Processor (e.g., L2100, L2200, U2400) line, which is capable of performing 500+MB/s of cryptographic instructions; VLSI Technology's 33 MHz 6868; and/or the like.

Memory

Generally, any mechanization and/or embodiment allowing a processor to affect the storage and/or retrieval of information is regarded as memory. However, memory is a fungible technology and resource, thus, any number of memory embodiments may be employed in lieu of or in concert with one another. It is to be understood that the controller of the present invention and/or a computer systemization may employ various forms of memory. For example, a computer systemization may be configured wherein the functionality of on-chip CPU memory (e.g., registers), RAM, ROM, and any other storage devices are provided by a paper punch tape or paper punch card mechanism; of course such an embodiment would result in an extremely slow rate of operation. In a typical configuration, memory will include ROM, RAM, and a storage device. A storage device may be any conventional computer system storage. Storage devices may include a drum; a (fixed and/or removable) magnetic disk drive; a magneto-optical drive; an optical drive (i.e., Blueray, CD ROM/RAM/Recordable (R)/ReWritable (RW), DVD R/RW, HD DVD R/RW etc.); an array of devices (e.g., Redundant Array of Independent Disks (RAID)); solid state memory devices (USB memory, solid state drives (SSD), etc.); other processor-readable storage mediums; and/or other devices of the like. Thus, a computer systemization generally requires and makes use of memory.

Component Collection

The memory may contain a collection of program and/or database components and/or data such as, but not limited to: operating system component(s) (operating system); information server component(s) (information server); user interface component(s) (user interface); Web browser component(s) (Web browser); database(s); mail server component(s); mail client component(s); cryptographic server component(s) (cryptographic server) and/or the like (i.e., collectively a component collection). These components may be stored and accessed from the storage devices and/or from storage devices accessible through an interface bus. Although non-conventional program components such as those in the component collection, typically, are stored in a local storage device, they may also be loaded and/or stored in memory such as: peripheral devices, RAM, remote storage facilities through a communications network, ROM, various forms of memory, and/or the like.

Operating System

The operating system component is an executable program component facilitating the operation of the controller of the present invention. Typically, the operating system facilitates access of I/O, network interfaces, peripheral devices, storage devices, and/or the like. The operating system may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/Millennium/NT/Vista/XP (Server), Palm OS, and/or the like. The operating system may be one specifically optimized to be run on a mobile computing device, such as iOS, Android, Windows Phone, Tizen, Symbian, and/or the like. An operating system may communicate to and/or with other components in a component collection, including itself, and/or the like. Most frequently, the operating system communicates with other program components, user interfaces, and/or the like. For example, the operating system may contain, communicate, generate, obtain, and/or provide program component, system, user, and/or data communications, requests, and/or responses. The operating system, once executed by the CPU, may enable the interaction with communications networks, data, I/O, peripheral devices, program components, memory, user input devices, and/or the like. The operating system may provide communications protocols that allow the controller of the present invention to communicate with other entities through a communications network. Various communication protocols may be used by the controller of the present invention as a subcarrier transport mechanism for interaction, such as, but not limited to: multicast, TCP/IP, UDP, unicast, and/or the like.

Information Server

An information server component is a stored program component that is executed by a CPU. The information server may be a conventional Internet information server such as, but not limited to Apache Software Foundation's Apache, Microsoft's Internet Information Server, and/or the like. The information server may allow for the execution of program components through facilities such as Active Server Page (ASP), ActiveX, (ANSI) (Objective-) C (++), C# and/or .NET, Common Gateway Interface (CGI) scripts, dynamic (D) hypertext markup language (HTML), FLASH, Java, JavaScript, Practical Extraction Report Language (PERL), Hypertext Pre-Processor (PHP), pipes, Python, wireless application protocol (WAP), WebObjects, and/or the like. The information server may support secure communications protocols such as, but not limited to, File Transfer Protocol (FTP); HyperText Transfer Protocol (HTTP); Secure Hypertext Transfer Protocol (HTTPS), Secure Socket Layer (SSL), messaging protocols (e.g., America Online (AOL) Instant Messenger (AIM), Application Exchange (APEX), ICQ, Internet Relay Chat (IRC), Microsoft Network (MSN) Messenger Service, Presence and Instant Messaging Protocol (PRIM), Internet Engineering Task Force's (IETF's) Session Initiation Protocol (SIP), SIP for Instant Messaging and Presence Leveraging Extensions (SIMPLE), open XML-based Extensible Messaging and Presence Protocol (XMPP) (i.e., Jabber or Open Mobile Alliance's (OMA's) Instant Messaging and Presence Service (IMPS)), Yahoo! Instant Messenger Service, and/or the like. The information server provides results in the form of Web pages to Web browsers, and allows for the manipulated generation of the Web pages through interaction with other program components. After a Domain Name System (DNS) resolution portion of an HTTP request is resolved to a particular information server, the information server resolves requests for information at specified locations on the controller of the present invention based on the remainder of the HTTP request. For example, a request such as http://123.124.125.126/myInformation.html might have the IP portion of the request “123.124.125.126” resolved by a DNS server to an information server at that IP address; that information server might in turn further parse the http request for the “/myInformation.html” portion of the request and resolve it to a location in memory containing the information “myInformation.html.” Additionally, other information serving protocols may be employed across various ports, e.g., FTP communications across port, and/or the like. An information server may communicate to and/or with other components in a component collection, including itself, and/or facilities of the like. Most frequently, the information server communicates with the database of the present invention, operating systems, other program components, user interfaces, Web browsers, and/or the like.

Access to the database of the present invention may be achieved through a number of database bridge mechanisms such as through scripting languages as enumerated below (e.g., CGI) and through inter-application communication channels as enumerated below (e.g., CORBA, WebObjects, etc.). Any data requests through a Web browser are parsed through the bridge mechanism into appropriate grammars as required by the present invention. In one embodiment, the information server would provide a Web form accessible by a Web browser. Entries made into supplied fields in the Web form are tagged as having been entered into the particular fields, and parsed as such. The entered terms are then passed along with the field tags, which act to instruct the parser to generate queries directed to appropriate tables and/or fields. In one embodiment, the parser may generate queries in standard SQL by instantiating a search string with the proper join/select commands based on the tagged text entries, wherein the resulting command is provided over the bridge mechanism to the present invention as a query. Upon generating query results from the query, the results are passed over the bridge mechanism, and may be parsed for formatting and generation of a new results Web page by the bridge mechanism. Such a new results Web page is then provided to the information server, which may supply it to the requesting Web browser.

Also, an information server may contain, communicate, generate, obtain, and/or provide program component, system, user, and/or data communications, requests, and/or responses.

User Interface

Computer interfaces in some respects are similar to automobile operation interfaces. Automobile operation interface elements such as steering wheels, gearshifts, and speedometers facilitate the access, operation, and display of automobile resources, and status. Computer interaction interface elements such as check boxes, cursors, menus, scrollers, and windows (collectively and commonly referred to as widgets) similarly facilitate the access, capabilities, operation, and display of data and computer hardware and operating system resources, and status. Operation interfaces are commonly called user interfaces. Graphical user interfaces (GUIs) such as the Apple Macintosh Operating System's Aqua, IBM's OS/2, Microsoft's Windows 2000/2003/3.1/95/98/CE/Millennium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, Yahoo! User Interface, any of which may be used and) provide a baseline and means of accessing and displaying information graphically to users.

A user interface component is a stored program component that is executed by a CPU. The user interface may be a conventional graphic user interface as provided by, with, and/or atop operating systems and/or operating environments such as already discussed. The user interface may allow for the display, execution, interaction, manipulation, and/or operation of program components and/or system facilities through textual and/or graphical facilities. The user interface provides a facility through which users may affect, interact, and/or operate a computer system. A user interface may communicate to and/or with other components in a component collection, including itself, and/or facilities of the like. Most frequently, the user interface communicates with operating systems, other program components, and/or the like. The user interface may contain, communicate, generate, obtain, and/or provide program component, system, user, and/or data communications, requests, and/or responses.

Web Browser

A Web browser component is a stored program component that is executed by a CPU. The Web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices. A Web browser may communicate to and/or with other components in a component collection, including itself, and/or facilities of the like. Most frequently, the Web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a Web browser and information server, a combined application may be developed to perform similar functions of both. The combined application would similarly affect the obtaining and the provision of information to users, user agents, and/or the like from the enabled nodes of the present invention. The combined application may be nugatory on systems employing standard Web browsers.

Mail Server

A mail server component is a stored program component that is executed by a CPU. The mail server may be a conventional Internet mail server such as, but not limited to sendmail, Microsoft Exchange, and/or the like. The mail server may allow for the execution of program components through facilities such as ASP, ActiveX, (ANSI) (Objective-) C (++), C# and/or .NET, CGI scripts, Java, JavaScript, PERL, PHP, pipes, Python, WebObjects, and/or the like. The mail server may support communications protocols such as, but not limited to: Internet message access protocol (IMAP), Messaging Application Programming Interface (MAPI)/Microsoft Exchange, post office protocol (POP3), simple mail transfer protocol (SMTP), and/or the like. The mail server can route, forward, and process incoming and outgoing mail messages that have been sent, relayed and/or otherwise traversing through and/or to the present invention.

Access to the mail of the present invention may be achieved through a number of APIs offered by the individual Web server components and/or the operating system.

Also, a mail server may contain, communicate, generate, obtain, and/or provide program component, system, user, and/or data communications, requests, information, and/or responses.

Mail Client

A mail client component is a stored program component that is executed by a CPU. The mail client may be a conventional mail viewing application such as Apple Mail, Microsoft Entourage, Microsoft Outlook, Microsoft Outlook Express, Mozilla, Thunderbird, and/or the like. Mail clients may support a number of transfer protocols, such as: IMAP, Microsoft Exchange, POP3, SMTP, and/or the like. A mail client may communicate to and/or with other components in a component collection, including itself, and/or facilities of the like. Most frequently, the mail client communicates with mail servers, operating systems, other mail clients, and/or the like; e.g., it may contain, communicate, generate, obtain, and/or provide program component, system, user, and/or data communications, requests, information, and/or responses. Generally, the mail client provides a facility to compose and transmit electronic mail messages.

Cryptographic Server

A cryptographic server component is a stored program component that is executed by a CPU, cryptographic processor, cryptographic processor interface, cryptographic processor device, and/or the like. Cryptographic processor interfaces will allow for expedition of encryption and/or decryption requests by the cryptographic component; however, the cryptographic component, alternatively, may run on a conventional CPU. The cryptographic component allows for the encryption and/or decryption of provided data. The cryptographic component allows for both symmetric and asymmetric (e.g., Pretty Good Protection (PGP)) encryption and/or decryption. The cryptographic component may employ cryptographic techniques such as, but not limited to: digital certificates (e.g., X.509 authentication framework), digital signatures, dual signatures, enveloping, password access protection, public key management, and/or the like. The cryptographic component will facilitate numerous (encryption and/or decryption) security protocols such as, but not limited to: checksum, Data Encryption Standard (DES), Elliptical Curve Encryption (ECC), International Data Encryption Algorithm (IDEA), Message Digest 5 (MD5, which is a one way hash function), passwords, Rivest Cipher (RCS), Rijndael, RSA (which is an Internet encryption and authentication system that uses an algorithm developed in 1977 by Ron Rivest, Adi Shamir, and Leonard Adleman), Secure Hash Algorithm (SHA), Secure Socket Layer (SSL), Secure Hypertext Transfer Protocol (HTTPS), and/or the like. Employing such encryption security protocols, the present invention may encrypt all incoming and/or outgoing communications and may serve as node within a virtual private network (VPN) with a wider communications network. The cryptographic component facilitates the process of “security authorization” whereby access to a resource is inhibited by a security protocol wherein the cryptographic component effects authorized access to the secured resource. In addition, the cryptographic component may provide unique identifiers of content, e.g., employing and MD5 hash to obtain a unique signature for an digital audio file. A cryptographic component may communicate to and/or with other components in a component collection, including itself, and/or facilities of the like. The cryptographic component supports encryption schemes allowing for the secure transmission of information across a communications network to enable the component of the present invention to engage in secure transactions if so desired. The cryptographic component facilitates the secure accessing of resources on the present invention and facilitates the access of secured resources on remote systems; i.e., it may act as a client and/or server of secured resources. Most frequently, the cryptographic component communicates with information servers, operating systems, other program components, and/or the like. The cryptographic component may contain, communicate, generate, obtain, and/or provide program component, system, user, and/or data communications, requests, and/or responses.

The Database of the Present Invention

The database component of the present invention may be embodied in a database and its stored data. The database is a stored program component, which is executed by the CPU; the stored program component portion configuring the CPU to process the stored data. The database may be a conventional, fault tolerant, relational, scalable, secure database such as Oracle or Sybase. Relational databases are an extension of a flat file. Relational databases consist of a series of related tables. The tables are interconnected via a key field. Use of the key field allows the combination of the tables by indexing against the key field; i.e., the key fields act as dimensional pivot points for combining information from various tables. Relationships generally identify links maintained between tables by matching primary keys. Primary keys represent fields that uniquely identify the rows of a table in a relational database. More precisely, they uniquely identify rows of a table on the “one” side of a one-to-many relationship.

Alternatively, the database of the present invention may be implemented using various standard data-structures, such as an array, hash, (linked) list, struct, structured text file (e.g., XML), table, and/or the like. Such data-structures may be stored in memory and/or in (structured) files. In another alternative, an object-oriented database may be used, such as Frontier, ObjectStore, Poet, Zope, and/or the like. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present invention is implemented as a data-structure, the use of the database of the present invention may be integrated into another component such as the component of the present invention. Also, the database may be implemented as a mix of data structures, objects, and relational structures. Databases may be consolidated and/or distributed in countless variations through standard data processing techniques. Portions of databases, e.g., tables, may be exported and/or imported and thus decentralized and/or integrated.

In one embodiment, the database component includes several tables. A Users (e.g., operators and physicians) table may include fields such as, but not limited to: user_id, ssn, dob, first_name, last_name, age, state, address_firstline, address_secondline, zipcode, devices_list, contact_info, contact_type, alt_contact_info, alt_contact_type, and/or the like to refer to any type of enterable data or selections discussed herein. The Users table may support and/or track multiple entity accounts. A Clients table may include fields such as, but not limited to: user_id, client_id, client_ip, client_type, client_model, operating_system, os_version, app_installed_flag, and/or the like. An Apps table may include fields such as, but not limited to: app_ID, app_name, app_type, OS_compatibilities_list, version, timestamp, developer_ID, and/or the like.

In one embodiment, user programs may contain various user interface primitives, which may serve to update the platform of the present invention. Also, various accounts may require custom database tables depending upon the environments and the types of clients the system of the present invention may need to serve. It should be noted that any unique fields may be designated as a key field throughout. In an alternative embodiment, these tables have been decentralized into their own databases and their respective database controllers (i.e., individual database controllers for each of the above tables). Employing standard data processing techniques, one may further distribute the databases over several computer systemizations and/or storage devices. Similarly, configurations of the decentralized database controllers may be varied by consolidating and/or distributing the various database components. The system of the present invention may be configured to keep track of various settings, inputs, and parameters via database controllers.

Various other components may be included and called upon for providing for aspects of the teachings herein. For example, additional materials, combinations of materials and/or omission of materials may be used to provide for added embodiments that are within the scope of the teachings herein. In the present application a variety of variables are described, including but not limited to components and conditions. It is to be understood that any combination of any of these variables can define an embodiment of the disclosure. Other combinations of articles, components, conditions, and/or methods can also be specifically selected from among variables listed herein to define other embodiments, as would be apparent to those of ordinary skill in the art.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method for managing healthcare information, comprising:
storing, in a memory, a data retrieval computer application comprising a fraud detection engine, a database of a plurality of healthcare providers, and a database of one or more users, wherein the database of one or more users includes a profile for each user of the one or more users;
enabling a user, using a graphical user interface (GUI), to search the database of the plurality of healthcare providers;
receiving, from the user, using the GUI, one or more search parameters via the graphical user interface;
returning, to the user, a list of all healthcare providers that satisfy the one or more search parameters;
selecting, using the GUI, a healthcare provider from the database of a plurality of healthcare providers that satisfy the one or more search parameters as a primary healthcare provider;
storing, in the memory, a patient care history of the user;
enabling, through the data retrieval computer application, remote access to a centralized information store by the user, the primary healthcare provider, and a third party insurer such that the fraud detection engine is configured to monitor medical claims of the user submitted to the third party insurer, wherein the centralized information store comprises pre-visit identifying documentation for the user;
enabling communication between the user, the primary healthcare provider, and the third party insurer using the data retrieval computer application, such that the fraud detection engine is configured to:
receive a new medical claim for the user submitted by the third party insurer;
transmit, in real-time, a claim verification message to the user for validation;
in response to receiving a confirmation of the new medical claim or one or more line items of the new medical claim from the user,
marking the new medical claim or the one or more line items of the new medical claim as validated; and
transmitting a verification notification of the new medical claim or the one or more line items of the new medical claim to the third party insurer for acceptance; and
in response to receiving a dispute of the new medical claim or the one or more line items of the new medical claim from the user,
marking the new medical claim or the one or more line items of the new medical claim as disputed; and
transmitting an audit notification to the third party insurer regarding the new medical claim or the one or more line items of the new medical claim.

2. The method as recited in claim 1,
wherein the search parameters are predefined, and
wherein the search parameters are selected from the group consisting of: a medical specialty, a name, a location, a telephone number, a provider type, languages spoken, and a type of insurance accepted.

3. The method as recited in claim 1,
wherein the database of the plurality of healthcare providers includes identifiable information pertaining to each of the plurality of healthcare providers selected from the group consisting of: a medical specialty, a name, a location, a telephone number, a provider type, languages spoken, and a type of insurance accepted, and
wherein the database of the one or more users includes identifiable information pertaining to each of the one or more users selected from the group consisting of: a name, an address, a telephone number, an insurance type, billing information, an employer, languages spoken, and a medical history.

4. The method as recited in claim 1, further comprising:
coordinating between the primary healthcare provider and the third party insurer, using the data retrieval computer application, to determine an insurance eligibility of the user for one or more medical procedures.

5. The method as recited in claim 1, further comprising:
enabling remote access to the user's insurance information and employment information to the user, the primary healthcare provider, and the third party insurer to facilitate a determination of insurance eligibility.

6. The method as recited in claim 1, further comprising:
enabling remote access to the user's insurance information and employment information to the user, the primary healthcare provider, and the third party insurer to facilitate payment of one or more medical procedures.

7. The method as recited in claim 1,
wherein the profile for each user of the one or more users includes any medical claims for that particular user, and
wherein the method further comprises: updating the medical claims for the user following approval or performance of a medical procedure.

8. The method as recited in claim 1,
wherein the profile for each user of the one or more users includes any outstanding payments due to the third party insurer for any medical procedures performed, and
wherein the method further comprises: automatically paying any outstanding balances using billing information provided by the user.

9. The method as recited in claim 1, wherein the enabling communication between the user, the primary healthcare provider, and the third party insurer using the data retrieval computer application further comprises enabling the user, the primary healthcare provider, and the third party insurer to communicate using a method selected from the group consisting of: e-mail; text message; video communication; and audio communication.

10. The method as recited in claim 1, further comprising:
enabling the user to designate one or more healthcare providers to a favorites list for future viewing.

11. A system for managing healthcare information, comprising:
a memory configured to store a data retrieval computer application comprising a fraud detection engine, a database of a plurality of healthcare providers, a database of one or more users, and a patient care history for each user, wherein the database of one or more users includes a profile for each user of the one or more users;
a graphical user interface (GUI); and
a processor configured to run the data retrieval computer application and access the database of a plurality of healthcare providers and the database of one or more users, wherein when the processor runs the data retrieval computer application, it causes a computing device to:
enable a user, using the GUI, to search the database of the plurality of healthcare providers;
receive, from the user using the GUI, one or more search parameters;
return, to the user, a list of all healthcare providers that satisfy the one or more search parameters;
accept, using the GUI, a healthcare provider, from the plurality of healthcare providers that satisfy the one or more search parameters as a primary healthcare provider;
enable remote access to a centralized information store by the user, the primary healthcare provider, and a third party insurer such that the fraud detection engine is configured to monitor medical claims of the user submitted to the third party insurer, wherein the centralized information store comprises pre-visit identifying documentation for the user;
enable communication between the user, the primary healthcare provider, and the third party insurer using the data retrieval computer application, such that the fraud detection engine is configured to:
receive a new medical claim for the user submitted by the third party insurer;
transmit, in real-time, a claim verification message to the user for validation;
in response to receiving a confirmation of the new medical claim or one or more line items of the new medical claim from the user,
marking the new medical claim or the one or more line items of the new medical claim as validated; and
transmitting a verification notification of the new medical claim or the one or more line items of the new medical claim to the third party insurer for acceptance; and
in response to receiving a dispute of the new medical claim or the one or more line items of the new medical claim from the user,
marking the new medical claim or the one or more line items of the new medical claim as disputed; and
transmitting an audit notification to the third party insurer regarding the new medical claim or the one or more line items of the new medical claim.

12. The system as recited in claim 11,
wherein the memory is further configured to store the one or more search parameters, and
wherein the one or more search parameters are selected from the group consisting of: a medical specialty, a name, a location, a telephone number, a provider type, languages spoken, and a type of insurance accepted.

13. The system as recited in claim 11, wherein the database of the plurality of healthcare providers includes identifiable information pertaining to each of the plurality of healthcare providers selected from the group consisting of: a medical specialty, a name, a location, a telephone number, a provider type, languages spoken, and a type of insurance accepted.

14. The system as recited in claim 11, wherein the database of the one or more users includes identifiable information pertaining to each of the one or more users selected from the group consisting of: a name, an address, a telephone number, an insurance type, billing information, an employer, languages spoken, and a medical history.

15. The system as recited in claim 11, wherein the processor is further configured to:
enable coordination between the primary healthcare provider and the third party insurer using the data retrieval computer application to determine an insurance eligibility of the user for one or more medical procedures.

16. The system as recited in claim 11, wherein the processor is further configured to:
enable remote access to the user's insurance information and employment information to the user, the primary healthcare provider, and the third party insurer to facilitate a determination of insurance eligibility.

17. The system as recited in claim 11, wherein the processor is further configured to:
enable remote access to the user's insurance information and employment information to the user, the primary healthcare provider, and the third party insurer to facilitate payment of one or more medical procedures.

18. The system as recited in claim 11,
wherein the profile for each user of the one or more users includes any medical claims for that particular user, and
wherein the processor is further configured to: update the medical claims for the particular user following an approval or a performance of a medical procedure.

19. The system as recited in claim 11, wherein the profile for each user of the one or more users includes any medical claims for that particular user and any outstanding payments due to the third party insurer for any medical procedures performed.

20. The system as recited in claim 11, wherein the processor is further configured to:
automatically pay any outstanding balances using billing information provided by the user.
